(19) United States
(12) Patent Application Publication
(10) Pub. No.: US 2026/0355444 A1
(43) Pub. Date: Nov. 12, 2026

(54) USER INTERFACE FOR CONTROLLING A RESPIRATORY APPARATUS

(71) Applicant: Fisher & Paykel Healthcare Limited, Auckland (NZ)

(72) Inventors: Matthew Robert WILSON, Auckland (NZ); Samantha Dale OLDFIELD, Auckland (NZ); Christopher Harding CAMPBELL, Auckland (NZ); Yijun REN, Auckland (NZ); Nathanael Charles GRAY, Auckland (NZ); Man Kit Jacky CHEUNG, Auckland (NZ); Matthew Jon PAYTON, Auckland (NZ); Hamish Adrian OSBORNE, Auckland (NZ)

(21) Appl. No.: 19/481,801

(22) PCT Filed: Jun. 7, 2024
(86) PCT No.: PCT/IB2024/055571

§ 371 Date: Nov. 5, 2025

Related U.S. Application Data
(60) Provisional application No. 63/507,257, filed on Jun. 9, 2023.

(51) Int. Cl. A61M 16/00; A61M 16/06; A61M 16/16

(52) U.S. Cl. CPC A61M 16/022; A61M 16/0066; A61M 16/0666; A61M 16/16; A61M 2202/0208; A61M 2205/18; A61M 2205/3334; A61M 2205/502

(57) ABSTRACT

A method for operating a respiratory apparatus comprises presenting on a display screen a display view comprising: a user-selectable first representation of a flow parameter which enables selection from a range of flow parameter values; and a user-selectable second representation of the flow parameter which comprises a single flow parameter set point value; receiving a user selection of the second representation; and responsive to the user selection, controlling the respiratory apparatus to provide a gases flow according to the parameter value corresponding to the second representation.

### TECHNICAL FIELD

The present invention relates to improvements in the operation and control of a respiratory apparatus providing a flow of gases to a patient. It relates specifically, but not exclusively, to methods, systems and computer program products providing user interfaces presenting views showing representations of flow parameters, that may drive simplified operation of the respiratory apparatus by a user interacting with the user interface.

### BACKGROUND OF INVENTION

Respiratory support may be provided to a patient in a number of different settings including intensive care units, hospital wards, during anaesthesia, or sedation, or more generally during certain medical procedures.

Respiratory support may be provided using a respiratory apparatus such as a ventilator, anaesthesia machine or other machines providing a flow of gas to the patient via a conduit and patient interface such as a nasal cannula, face mask, laryngeal mask airway (LMA) or the like. Some machines can have a processor controlled display device which presents to the user prompts and control parameters relating to the respiratory support being provided. It would be useful to provide one or more improvements to the manner in which a respiratory apparatus may be controlled and/or operated for the provision of respiratory support to a patient.

The discussion of the background to the invention included herein including reference to documents, acts, materials, devices, articles and the like is included to explain the context of the present invention. This is not to be taken as an admission or a suggestion that any of the material referred to was published, known or part of the common general knowledge.

### SUMMARY OF INVENTION

Viewed from one aspect, the present disclosure provides a method for operating a respiratory apparatus, comprising the steps of: presenting on a display screen a display view comprising: a user-selectable first representation of a flow parameter, the first representation enabling selection from a range of flow parameter values; and a user-selectable second representation of the flow parameter, the second representation comprising a single flow parameter set point value; receiving a user selection of the second representation; and responsive to the user selection, controlling the respiratory apparatus to provide a gases flow according to the parameter value corresponding to the second representation.

In some embodiments, the method may comprise receiving a selection of the first representation and, responsive to that user selection, controlling the respiratory apparatus to provide a gases flow according to the user's selected value from the range corresponding to the first representation.

In some embodiments, the user-selectable first representation and the user-selectable second representation are discrete representations.

In some embodiments, the user-selectable first representation and the user-selectable second representation are presented on the display view substantially simultaneously.

In some embodiments, the respiratory apparatus comprises at least one of a blower and a valve, and wherein the method comprises controlling the blower and/or the valve to provide the gases flow according to the parameter value corresponding to the second representation.

In some embodiments, the user selection of the second representation is received via the display screen, and the respiratory apparatus is controlled to provide the gases flow according to the parameter value corresponding to the second representation.

In some embodiments, the gases flow is provided to a patient via a non-sealing interface that is in fluid communication with an outlet of the respiratory apparatus.

In some embodiments, the flow parameter comprises flow rate. In some embodiments, the method comprises providing the gases flow at a flow rate of at least about 15 LPM. In some embodiments, the second representation comprises a flow rate set point value selected from the group comprising but not limited to 40 LPM and 70 LPM.

In some embodiments, the method comprises presenting on the display view a third user-selectable representation of the flow parameter, the third representation comprising a single flow parameter set point value different from the set point value of the second representation. In some embodiments, the third representation comprises a flow rate set point value selected from the group comprising but not limited to 40 LPM and 70 LPM.

In some embodiments, the second representation and the third representation each comprise a single input selector. The third representation may be a discrete representation from the first and second representations.

In some embodiments, the third representation is presented substantially simultaneously with the first and second representations.

In some embodiments, the first representation comprises one or more of a slider; a dial; up/down arrows; +/− symbols; other symbols representing an increase or decrease to the flow parameter value within the range.

In some embodiments, the display screen comprises a touch screen.

In some embodiments, the user selections of the representations presented in the display view are received by a controller which, responsive to the user selections, controls the respiratory apparatus.

In some embodiments, the method comprises upon startup of the apparatus, the controller setting a flow parameter for the gases flow to a predetermined oxygen concentration value. The method may comprise, responsive to the controller receiving a user selection of a flow rate value, controlling the respiratory apparatus to provide the gases flow at the predetermined oxygen concentration value and the selected flow rate value. The predetermined oxygen concentration value may be e.g. 21% FiO2 or 100% FiO2.

In some embodiments, the method comprises upon startup of the respiratory apparatus, presenting on the display view a user-selectable further representation of a predetermined oxygen concentration value for the gases flow; and optionally wherein the predetermined oxygen concentration value be e.g. 21% FiO2 or 100% FiO2.

In some embodiments, the controller does not prompt the user for a confirmation input when controlling the respiratory apparatus to provide a gases flow according to a flow rate value corresponding to user selection of any one of the first representation, the second representation and the third representation.

Viewed from another aspect, the present disclosure provides a method for operating a respiratory apparatus, comprising the steps of: presenting on a display screen a display view comprising: a user-selectable first representation of a flow parameter, the first representation enabling selection from a range of flow parameter values; and a user-selectable second representation of the flow parameter, the second representation comprising a single flow parameter set point value; receiving a user selection of the first representation or the second representation; and responsive to the user selection, controlling the respiratory apparatus to provide a gases flow according to the parameter value corresponding to the selected first representation or the selected second representation.

In some embodiments, wherein the user-selectable first representation and the user-selectable second representation are discrete representations.

In some embodiments, the user-selectable first representation and the user-selectable second representation are presented on the display view substantially simultaneously.

In some embodiments, the respiratory apparatus comprises at least one of a blower and a valve, and wherein the method comprises controlling the blower and/or the valve to provide the gases flow according to the parameter value corresponding to the selected first representation or the selected second representation.

In some embodiments, the user selection of the first representation or the second representation is received via the display screen, and the respiratory apparatus is controlled to provide the gases flow according to the parameter value corresponding to the selected first representation or the selected second representation.

In some embodiments, the gases flow is provided to a patient via a non-sealing interface that is in fluid communication with an outlet of the respiratory apparatus.

In some embodiments, the flow parameter comprises flow rate.

In some embodiments, the method comprises providing the gases flow at a flow rate of at least about 15 LPM.

In some embodiments, the second representation comprises a flow rate set point value selected from the group comprising 40 LPM and 70 LPM.

In some embodiments, the method comprises presenting on the display view a third user-selectable representation of the flow parameter, the third representation comprising a single flow parameter set point value different from the set point value of the second representation. In some embodiments, the second representation and the third representation each comprise a single input selector. The third representation may comprise a flow rate set point value selected from the group comprising 40 LPM and 70 LPM. In some embodiments, the third representation is a discrete representation from the first and second representations. The third representation may be presented substantially simultaneously with the first and second representations.

In some embodiments, the first representation comprises one or more of a slider; a dial; up/down arrows; +/− symbols; other symbols representing an increase or decrease to the flow parameter value within the range.

In some embodiments, the display screen comprises a touch screen.

In some embodiments, the user selections of the representations presented in the display view are received by a controller which, responsive to the user selections, controls the respiratory apparatus.

In some embodiments, the method comprises, upon startup of the apparatus, the controller setting a flow parameter for the gases flow to a predetermined oxygen concentration value. In some embodiments the method comprises, responsive to the controller receiving a user selection of a flow rate value, controlling the respiratory apparatus to provide the gases flow at the predetermined oxygen concentration value and the selected flow rate value. The predetermined oxygen concentration value may be e.g. 21% FiO2 or 100% FiO2.

In some embodiments, the controller does not prompt the user for a confirmation input when controlling the respiratory apparatus to provide a gases flow according to a flow rate value corresponding to user selection of any one of the first representation, the second representation and the third representation.

An aspect of the disclosure provides a system for operating a respiratory apparatus according to the methods disclosed above.

Viewed from another aspect, the present disclosure provides a system for operating a flow source, the system comprising: a display screen operable to present a display view comprising: a user-selectable first representation of a flow parameter, the first representation enabling selection from a range of flow parameter values; and a user-selectable second representation of the flow parameter, the second representation comprising a single flow parameter set point value; and a controller for receiving user selections, and responsive to a user selection of the first representation or the second representation, controlling a flow source to provide the gases flow according to the parameter value corresponding to the selected first representation or the selected second representation.

In some embodiments, the system comprises the flow source.

The flow source may comprise at least one of a blower and a valve and the controller controls the blower and/or the valve to provide the gases flow according to the parameter value corresponding to the first representation or the second representation.

In some embodiments, the user selection of the first representation or the second representation is received at the controller via the display screen, and the controller controls the flow source to provide the gases flow according to the parameter set point value corresponding to the first representation or the second representation.

In some embodiments of the system, a selection of the first representation is received at the controller and, responsive to that user selection, the controller controls the respiratory apparatus to provide a gases flow according to the user's selected value from the range corresponding to the first representation.

In some embodiments of the system, the gases flow is provided to the patient via a non-sealing interface that is provided in fluid communication with an outlet of the flow source.

In some embodiments of the system, the flow parameter comprises flow rate.

The controller may control the flow source to provide the gases flow at a flow rate of at least about 15 LPM.

In some embodiments of the system, the second representation comprises a flow rate set point value selected from the group comprising but not limited to 40 LPM and 70 LPM.

In some embodiments of the system, the display screen is operable to present on the display view a third user-selectable representation of the flow parameter, the third representation comprising a single flow parameter set point value different from the set point value of the second representation. The second representation and the third representation may each comprise a single input selector. The third representation may comprise a flow rate set point value selected from the group comprising but not limited to 40 LPM and 70 LPM. The third representation may be presented substantially simultaneously with the first and second representations.

In some embodiments of the system, the first representation comprises one or more of a slider; a dial; up/down arrows; +/− symbols; other symbols representing an increase or decrease to the flow parameter value within the range.

In some embodiments of the system, the display screen comprises a touch screen.

In some embodiments of the system, the flow source comprises one or more gases inlets. In some embodiments, the system comprises one or more gas outlets for fluid communication with a conduit configured to provide the gases flow to the patient. The one or more gas outlets may be couplable with a conduit configured to provide the gases flow to a patient interface.

The system may comprise a humidifier.

In some embodiments, the system is operable to control a flow source provided in one or both of: a separate device; and a respiratory apparatus incorporating the system comprising the display screen and the controller.

In some embodiments, the system comprises part of an anaesthesia machine with capability to provide high flow gases, and the controller controls the gases flow from the anaesthesia machine according to user selections of one or more flow parameter values received by the controller.

In some embodiments, the system comprises part of a respiratory apparatus providing the gases flow according to user selections of one or more flow parameter values received by the controller.

In some embodiments, the system comprises part of an anaesthesia machine or ventilator, wherein the controller is operatively couplable with a respiratory apparatus separate from the anaesthesia machine or ventilator and which is operable to provide the gases flow according to user selections of one or more flow parameter values received by the controller. The anaesthesia machine or ventilator may be operatively couplable with the respiratory apparatus by one or more of a wireless, wired or contact coupling. The anaesthesia machine or ventilator may comprise a physical interface configured to cooperate with the respiratory apparatus. In some embodiments, the physical interface may comprise a recess for receiving at least part of the respiratory apparatus.

In some embodiments, upon startup of the system, the controller sets a flow parameter for the gases flow to a predetermined oxygen concentration value. Responsive to receiving a user selection of a flow rate value following startup, the controller may control the flow source to provide the gases flow at the predetermined oxygen concentration value and the selected flow rate value. The predetermined oxygen concentration value may be e.g. 21% FiO2 or 100% FiO2.

In some embodiments, upon startup of the system, the controller causes presentation on the display view of a user-selectable further representation of a predetermined oxygen concentration value for the gases flow. The predetermined oxygen concentration value may be, for example, 21% FiO2 or 100% FiO2.

In some embodiments of the system, the controller does not prompt the user for a confirmation input when controlling the respiratory apparatus to provide a gases flow according to a flow rate value corresponding to user selection of any one of the first representation, the second representation and the third representation.

An aspect of the disclosure provides a computer program product embodied on a memory device containing instructions for implementing, when executed by a controller, the method disclosed above.

Viewed from another aspect, the present disclosure provides a computer program product embodied on a memory device containing instructions causing a controller having an associated display screen to: perform a method for operating a respiratory apparatus, comprising: presenting on the display screen a display view comprising: a user-selectable first representation of a flow parameter, the first representation enabling selection from a range of flow parameter values; and a user-selectable second representation of the flow parameter, the second representation comprising a single flow parameter set point value; receiving at the controller, a user selection of the second representation; and responsive to the user selection, the controller controlling the respiratory apparatus to provide a gases flow according to the parameter value corresponding to the second representation.

In some embodiments of the computer program product, the user-selectable first representation and the user-selectable second representation are discrete representations. The user-selectable first representation and the user-selectable second representation may be presented on the display view substantially simultaneously.

In some embodiments of the computer program product, the instructions may cause the controller to perform the method comprising receiving a selection of the first representation and, responsive to that user selection, controlling the respiratory apparatus to provide a gases flow according to the user's selected value from the range corresponding to the first representation.

In some embodiments of the computer program product, the respiratory apparatus operated according to the method comprises at least one of a blower and a valve, and wherein the method comprises the controller controlling the blower and/or the valve to provide the gases flow according to the parameter value corresponding to the second representation.

In some embodiments of the computer program product, the flow parameter comprises flow rate. The instructions may cause the controller to perform the method comprising providing the gases flow at a flow rate of at least about 15 LPM.

In some embodiments of the computer program product, the second representation comprises a flow rate set point value selected from the group comprising but not limited to 40 LPM and 70 LPM.

In some embodiments of the computer program product, the instructions may cause the controller to perform the method comprising presenting on the display view a user-selectable third representation of the flow parameter, the third representation comprising a single flow parameter set point value different from the value of the second representation. The third representation may comprise a flow rate set point value selected from the group comprising but not limited to 40 LPM and 70 LPM.

In some embodiments of the computer program product, the second representation and the third representation each comprise a single input selector. The third representation may be a discrete representation from the first and second representations. The third representation may be presented substantially simultaneously with the first and second representations.

In some embodiments of the computer program product, the first representation comprises one or more of a slider; a dial; up/down arrows; +/− symbols; other paired symbols representing an increase or decrease to the flow parameter value within the range.

In some embodiments of the computer program product, the instructions cause the controller to control a display screen comprising a touch screen.

In some embodiments of the computer program product, the instructions cause the controller to perform the method comprising presenting on the display view the first representation in a font size that is less than the font size of one or both of the second representation and the third representation.

In some embodiments of the computer program product, the instructions cause the controller to perform the method comprising presenting on the display view all of the first representation, the second representation and the third representation in a common colour and/or rendering.

In some embodiments of the computer program product, the instructions cause the controller to perform the method comprising presenting on the display view a user-selectable fourth representation of a second flow parameter, the fourth representation enabling selection from a range of second flow parameter values. The fourth representation may comprise one or more of a slider; a dial; up/down arrows; +/− symbols; other paired symbols representing an increase or decrease to the second flow parameter value within the range. The fourth representation may comprise e.g. Fraction of Inspired Oxygen (FiO2).

Viewed from another aspect, the present disclosure provides a method for controlling a respiratory apparatus, comprising, presenting on a display screen a display view comprising a representation of a flow parameter setting; controlling the respiratory apparatus to provide the gases flow according to the flow parameter setting; receiving values representing a flow parameter of the gases flow; and responsive to receiving a system-generated trigger, automatically causing the display view to replace the presented representation of the flow parameter setting with presentation of the received values representing the flow parameter of the gases flow.

In some embodiments, the system-generated trigger comprises the received values crossing a pre-determined threshold. The pre-determined threshold may comprise at least about +/−5% of the flow parameter setting. Alternatively or additionally, the pre-determined threshold may comprise at least about +/−10% of the flow parameter setting.

In some embodiments, the system-generated trigger comprises a mis-match between received values representing the flow parameter and a pre-determined relationship.

In some embodiments, the system-generated trigger comprises a physiological indicator determined by one or more instruments monitoring one or more patient condition parameters. The one or more patient condition parameters may be selected from a group comprising but not limited to: a blood gas parameter; an expired gas parameter; an inspired gas parameter; and patient position.

In some embodiments, the system-generated trigger comprises a breathing interface indicator determined by instrumented detection of a mask placed over a nasal cannula providing a gases flow to a patient. The breathing interface indicator may be triggered by one or more instruments monitoring parameters selected from a group comprising but not limited to acoustic parameters; proximity parameters; pressure parameters; gas concentration parameters; and optical parameters.

In some embodiments, the system-generated trigger may be triggered by an instrumented (e.g. non-human initiated) input. In some embodiments, an instrumented input may represent one or more of a condition, state, observation or performance measure of an aspect of the respiratory apparatus and/or the patient. In some embodiments, system-generated triggers do not comprise time-based triggers solely activated by a clock, timer, time delay or time-based measure in the absence of some other system-generated non-time based input.

In some embodiments the method comprises, responsive to receiving the system-generated trigger, automatically causing an audible device to present an audible and/or visible alert.

In some embodiments the method comprises, responsive to receiving the system-generated trigger, automatically causing a colour and/or brightness change on at least a part of the display view.

In some embodiments, the received values represent the same flow parameter as the flow parameter setting. The flow parameter may comprise flow rate. The flow parameter may comprise Fraction of Inspired Oxygen (FiO2). The flow parameter may comprise pressure of gases in the gases flow.

In some embodiments the method comprises, responsive to a controller receiving the system-generated trigger, the controller executing a delay routine before automatically causing the display view to replace the presented flow parameter setting with presentation of the received values.

In some embodiments, the system-generated trigger is determined according to a mode of operation of the respiratory apparatus.

In some embodiments, the system-generated trigger is determined according to whether the respiratory apparatus is operating in a pressure-controlled mode or a flow-controlled mode.

In some embodiments, the received values are generated by a controller receiving a signal from one or more sensors and the controller, responsive to determining that the system-generated trigger has been received, automatically causes the display view to replace the representation of the flow parameter setting with the received values representing the flow parameter of the gases flow.

In some embodiments the method comprises, responsive to the controller determining that the system-generated trigger has been received, the controller causing changes to the representation on the display view comprising one or more of a colour change in the representation, a size change in the representation, a change in textural rendering in the representation, and display and/or sounding of an alert.

An aspect of the disclosure provides a system for controlling a respiratory apparatus according to the method disclosed above.

Viewed from another aspect, the present disclosure provides a system for controlling a respiratory apparatus, the system comprising: a display screen operable to present a display view comprising: a representation of a flow parameter setting; a controller for: controlling the respiratory apparatus to provide the gases flow according to the flow parameter setting; determining received values representing a flow parameter of the gases flow; and responsive to receiving a system-generated trigger, automatically causing the display view to replace the presented representation of the flow parameter setting with presentation of the received values representing the flow parameter of the gases flow.

In some embodiments of the system, the system-generated trigger comprises the received values crossing a pre-determined threshold. The pre-determined threshold may be at least about +/−5% of the flow parameter setting. Alternatively or additionally, the pre-determined threshold may comprise at least about +/−10% of the flow parameter setting.

In some embodiments of the system, the system-generated trigger comprises a mis-match between received values representing the flow parameter and a pre-determined relationship.

In some embodiments of the system, the system-generated trigger comprises a physiological indicator determined by one or more instruments monitoring one or more patient condition parameters. The one or more patient condition parameters may be selected from a group comprising but not limited to a blood gas parameter; an expired gas parameter; an inspired gas parameter; and patient position.

In some embodiments of the system, the system-generated trigger comprises a breathing interface indicator determined by instrumented detection of a mask placed over a nasal cannula providing a gases flow to a patient. The breathing interface indicator may be triggered by one or more instruments monitoring parameters selected from a group comprising but not limited to acoustic parameters; proximity parameters; pressure parameters; gas concentration parameters; and optical parameters.

In some embodiments of the system, the system-generated trigger may be triggered by an instrumented (e.g. non-human initiated) input. In some embodiments, an instrumented input may represent one or more of a condition, state, observation or performance measure of an aspect of the respiratory apparatus and/or the patient. In some embodiments, system-generated triggers do not comprise time-based triggers solely activated by a clock, timer, time delay or time-based measure in the absence of some other system-generated non-time based input.

In some embodiments of the system, the controller, responsive to receiving the system-generated trigger, automatically causes an audible device to present an audible and/or visible alert.

In some embodiments of the system, the controller, responsive to receiving the system-generated trigger, automatically causes a colour and/or brightness change on at least a part of the display view.

In some embodiments of the system, the received values represent the same flow parameter as the flow parameter setting. The flow parameter may comprise flow rate. The flow parameter may comprise Fraction of Inspired Oxygen (FiO2). The flow parameter may comprise pressure of gases in the gases flow.

In some embodiments of the system, responsive to the controller receiving the system-generated trigger, the controller may execute a delay routine before automatically causing the display view to replace the presented flow parameter setting with presentation of the received values.

In some embodiments of the system, the controller determines the system-generated trigger according to a mode of operation of the respiratory apparatus.

In some embodiments of the system, the controller determines the system-generated trigger according to whether the respiratory apparatus is operating in a pressure-controlled mode or a flow-controlled mode.

In some embodiments of the system, the controller determines the received values from signals received from one or more sensors and/or components of the respiratory apparatus.

In some embodiments, the system comprises the respiratory apparatus. The respiratory apparatus may comprise a flow source. In some embodiments, the system comprises one or more gases inlets to the respiratory apparatus. In some embodiments, the system comprises one or more gas outlets for fluid communication with a conduit configured to provide the gases flow to the patient. The one or more gas outlets may be couplable with a conduit configured to provide the gases flow to a patient interface.

In some embodiments of the system, the display screen comprises a touch screen.

The system may comprise a humidifier.

In some embodiments, the system is operable to control a flow source provided in one or both of a separate device; and a respiratory apparatus incorporating the system comprising the display screen and the controller.

In some embodiments, the system comprises part of an anaesthesia machine with capability to provide high flow gases, wherein the controller controls the gases flow from the anaesthesia machine according to the flow parameter setting.

In some embodiments, the system comprises part of a respiratory apparatus providing the gases flow according to the flow parameter setting.

In some embodiments, the system comprises part of an anaesthesia machine or ventilator, wherein the controller is operatively couplable with a respiratory apparatus separate from the anaesthesia machine or ventilator and which is operable to provide the gases flow according to the flow parameter setting. The anaesthesia machine or ventilator may be operatively couplable with the respiratory apparatus by one or more of a wireless, wired or contact coupling. The anaesthesia machine or ventilator may comprise a physical interface configured to cooperate with the respiratory apparatus. In some embodiments, the physical interface may comprise a recess for receiving at least part of the respiratory apparatus.

An aspect of the disclosure provides a computer program product embodied on a memory device containing instructions for implementing, when executed by a controller, the method disclosed above.

Viewed from another aspect, the present disclosure provides a computer program product embodied on a memory device containing instructions causing a controller having an associated display screen to: perform a method for operating a respiratory apparatus, comprising: presenting on the display screen a display view comprising a representation of a flow parameter setting; controlling the respiratory apparatus to provide the gases flow according to the flow parameter setting; determining received values representing a flow parameter of the gases flow; and responsive to receiving a system-generated trigger, automatically causing the display view to replace the presented representation of the flow parameter setting with presentation of the received values representing the flow parameter of the gases flow.

In some embodiments of the computer program product, the system-generated trigger comprises the received values crossing a pre-determined threshold. The pre-determined threshold may comprise at least about +/−5% of the flow parameter setting.

Alternatively or additionally, the pre-determined threshold may comprise at least about +/−10% of the flow parameter setting.

In some embodiments of the computer program product, the system-generated trigger comprises a mis-match between received values representing the flow parameter and a pre-determined relationship.

In some embodiments of the computer program product, the system-generated trigger comprises a physiological indicator determined by one or more instruments monitoring one or more patient condition parameters. The one or more patient condition parameters may be selected from a group comprising but not limited to a blood gas parameter; an expired gas parameter; an inspired gas parameter; and patient position.

In some embodiments of the computer program product, the system-generated trigger comprises a breathing interface indicator determined by instrumented detection of a mask placed over a nasal cannula providing a gases flow to a patient. The breathing interface indicator may be triggered by one or more instruments monitoring parameters selected from a group comprising but not limited to acoustic parameters; proximity parameters; pressure parameters; gas concentration parameters; and optical parameters.

In some embodiments of the computer program product, the system-generated trigger may be triggered by an instrumented (e.g. non-human initiated) input. In some embodiments, an instrumented input may represent one or more of a condition, state, observation or performance measure of an aspect of the respiratory apparatus and/or the patient. In some embodiments, system-generated triggers do not comprise time-based triggers solely activated by a clock, timer, time delay or time-based measure in the absence of some other system-generated non-time based input.

In some embodiments of the computer program product, the instructions cause the controller to, responsive to receiving the system-generated trigger, automatically cause an audible device to present an audible and/or visible alert.

In some embodiments of the computer program product, the instructions cause the controller to, responsive to receiving the system-generated trigger, automatically cause a colour and/or brightness change on at least a part of the display view.

In some embodiments of the computer program product, the received values represent the same flow parameter as the flow parameter setting. The flow parameter may comprise flow rate. The flow parameter may comprise Fraction of Inspired Oxygen (FiO2).

The flow parameter may comprise pressure of gases in the gases flow.

In some embodiments of the computer program product, the instructions cause the controller to, responsive to the controller receiving the system-generated trigger, execute a delay routine before automatically causing the display view to replace the presented flow parameter setting with presentation of the received values.

In some embodiments of the computer program product, the system-generated trigger is determined according to a mode of operation of the respiratory apparatus.

In some embodiments of the computer program product, the system-generated trigger is determined according to whether the respiratory apparatus is operating in a pressure-controlled mode or a flow-controlled mode.

In some embodiments of the computer program product, the instructions cause the controller to determine the received values according to a signal received from one or more sensors.

Viewed from another aspect, the present disclosure provides a respiratory support apparatus comprising: a controller; and a display device comprising a display screen; wherein the controller controls the display device to present on the display screen a user interface/display view for selecting/adjusting a flow parameter of the respiratory support; and wherein the user interface/display view comprises: a user-selectable first representation of a flow parameter, the first representation enabling selection from a range of flow parameter values; and a user-selectable second representation of the flow parameter, the second representation comprising a single flow parameter set point value.

In some embodiments, the controller controls the respiratory apparatus to provide a flow of gases according to the parameter value corresponding to a user selection received by the controller.

It is to be understood each of the various aspects described herein may incorporate one or more features, modifications and alternatives described in the context of one or more other aspects and may include one or more features, modifications and alternatives of any of the embodiments described below, as appropriate. For efficiency, such features, modifications and alternatives have not been repetitiously disclosed for each and every aspect although one of skill in the art will appreciate that such combinations of features, modifications and alternatives disclosed for some aspects and embodiments apply similarly for other aspects and are within the scope of and form part of the subject matter of this disclosure.

### BRIEF DESCRIPTION OF DRAWINGS

Embodiments of the disclosure will now be described in greater detail with reference to the following Figures. It is to be understood that the embodiments shown are examples only and are not to be taken as limiting the scope of the invention as defined in the claims appended hereto.

FIG. 1 is a schematic diagram of an example of a respiratory system for providing respiratory gases to a patient.

FIG. 2 shows a patient wearing a patient interface comprising a non-sealing nasal cannula.

FIG. 3 is a flow chart representing a method for controlling a respiratory apparatus, providing user-selectable representations on a display view for quick selection of flow parameters to be provided by the respiratory apparatus.

FIG. 4 is an example of a display view relating to the method of FIG. 3.

FIG. 5 is another example of a display view relating to the method of FIG. 3.

FIG. 6 is an example of a display view relating to the method of FIG. 3 in which a flow rate has been selected.

FIG. 7 is an example of a display view presenting a confirmation prompt to confirm a flow rate change to stop flow.

FIG. 8 is a flow chart representing a method for controlling a respiratory apparatus, providing a display view that switches from presenting flow parameter values corresponding to “set points” to flow parameter values representing a parameter of the actual flow, when a system-generated trigger is received.

FIG. 9 is an example of a display view relating to the method of FIG. 8.

FIG. 10 is another example of a display view relating to the method of FIG. 8.

FIG. 11 is another example of a display view relating to the method of FIG. 8.

FIG. 12 is an example of a display view containing an alert relating to O2 supply failure.

FIG. 13 is an example of a display view presenting a warning view and guide for troubleshooting an O2 supply failure.

FIG. 14 is an example of a display view presenting a confirmation prompt to confirm an O2 supply failure exists.

FIG. 15 is an example of a display view presenting a warning view and guide for troubleshooting a patient disconnection error.

FIG. 16 is an example of a display view presenting the user-selected operational parameters.

FIGS. 17 to 19 are schematic representations showing alternative layouts for display views according to the present disclosure.

FIG. 20 is an example of a display device comprising a transient display screen and non-transient buttons on the display device.

FIG. 21 is an example of a display view according to another embodiment of the disclosure providing finer control over selections made by the user.

FIG. 22 is an example of a display view according to another embodiment of the disclosure alerting the user to the selection of FiO2 being a high value using an icon.

FIG. 23 is an example of a display view according to another embodiment of the disclosure alerting the user to the selection of FiO2 being a high value using an outline.

FIGS. 24 and 25 show a patient wearing a first patient interface (FIG. 24) and a first patient interface with a second patient interface (FIG. 25) for use with a respiratory system for providing respiratory gases to a patient according to an embodiment of the present disclosure.

FIGS. 26 and 27 are schematic illustrations of the first state and second state of a collapsible portion of the patient interface; FIG. 26 shows the first state and FIG. 27 shows the second state.

### DETAILED DESCRIPTION

Throughout the figures and specification, similar reference numerals may be used to designate the same or similar features, and descriptions thereof may be omitted or limited for simplicity and conciseness. It is to be understood that the description of features in relation to one embodiment is, unless stated otherwise, considered relevant to understanding the same or similar features in relation to other embodiments.

As mentioned, respiratory systems provide gas to a patient. Respiratory systems may take a number of forms, such as positive airway pressure systems (PAP) and high flow respiratory gas systems (e.g. for use in high flow therapy and anaesthesia procedures).

In this specification, “high flow” means, without limitation, any gas flow with a flow rate that is higher than usual/normal, such as higher than the normal inspiration flow rate of a healthy patient. It can be provided by a non-sealing respiratory system with uncontrolled and often substantial leaks happening at the entrance of the patient's airways due to a non-sealing patient interface, for example non-sealing nasal prongs. It can be also provided with humidification to improve patient comfort, compliance and safety.

Alternatively or additionally, it can be higher than some other threshold flow rate that is relevant to the context-for example, where providing a gas flow to a patient at a flow rate to meet inspiratory demand (e.g. instantaneous inspiratory demand or peak inspiratory demand-this could be the inspiratory demand of the patient that is receiving the respiratory support, or a representative inspiratory demand, such as representative of patients based on e.g. empirical data), that flow rate might be deemed “high flow” as it is higher than a nominal flow rate that might have otherwise been provided. “High flow” is therefore context dependent, and what constitutes “high flow” depends on many factors such as the health state of the patient, the type of procedure/therapy/support being provided, the nature of the patient (big, small, adult, child) and the like. Those skilled in the art would appreciate from context what constitutes “high flow”. It is a magnitude of flow rate that is over and above a flow rate that might otherwise be provided.

But, without limitation, some indicative values of high flow can be as follows.

In some configurations, the provision of gases to a patient at a flow rate of greater than or equal to about 5 or 10 litres per minute (5 or 10 LPM or L/min).

In some configurations, provision of gases to a patient at a flow rate of about 5 or 10 LPM to about 150 LPM, or about 15 LPM to about 95 LPM, or about 20 LPM to about 90 LPM, or about 25 LPM to about 85 LPM, or about 30 LPM to about 80 LPM, or about 35 LPM to about 75 LPM, or about 40 LPM to about 70 LPM, or about 45 LPM to about 65 LPM, or about 50 LPM to about 60 LPM. For example, according to various embodiments and configurations described herein, a flow rate of gases supplied or provided to an interface via a system or from a flow source or flow modulator, may comprise, but is not limited to, flows of at least about 5, 10, 20, 30, 40, 50, 60, 70, 80, 90, 100, 110, 120, 130, 140, 150 LPM, or more, and useful ranges may be selected to be any of these values (for example, about 20 LPM to about 90 LPM, about 40 LPM to about 70 LPM, about 40 LPM to about 80 LPM, about 50 LPM to about 80 LPM, about 60 LPM to about 80 LPM, about 70 LPM to about 100 LPM, about 70 LPM to about 80 LPM).

In “high flow” the gas provided will be chosen depending on for example the intended use of a therapy. Gases provided may comprise a percentage of oxygen. In some configurations, the percentage of oxygen in the gases provided may be about 15% to about 100%, 20% to about 100%, or about 30% to about 100%, or about 40% to about 100%, or about 50% to about 100%, or about 60% to about 100%, or about 70% to about 100%, or about 80% to about 100%, or about 90% to about 100%, or about 100%, or 100%.

In some embodiments, gases provided may comprise a percentage of carbon dioxide. In some configurations, the percentage of carbon dioxide in the gases provided may be more than 0%, about 0.3% to about 100%, about 1% to about 100%, about 5% to about 100%, about 10% to about 100%, about 20% to about 100%, or about 30% to about 100%, or about 40% to about 100%, or about 50% to about 100%, or about 60% to about 100%, or about 70% to about 100%, or about 80% to about 100%, or about 90% to about 100%, or about 100%, or 100%.

Flow rates for “High flow” for premature/infants/paediatrics (with body mass in the range of about 1 to about 30 kg) can be different. The flow rate can be set to 0.4-8 L/min/kg with a minimum of about 0.5 L/min and a maximum of about 70 L/min. For patients under 2 kg maximum flow may be set to 8 L/min.

High flow has been found effective in meeting or exceeding the patient's normal inspiratory flow, to increase oxygenation of the patient and/or reduce the work of breathing. Additionally, high flow therapy may generate a flushing effect in the nasopharynx such that the anatomical dead space of the upper airways is flushed by the high incoming gas flows. This creates a reservoir of fresh gas available for each and every breath, while minimising re-breathing of carbon dioxide, nitrogen, etc.

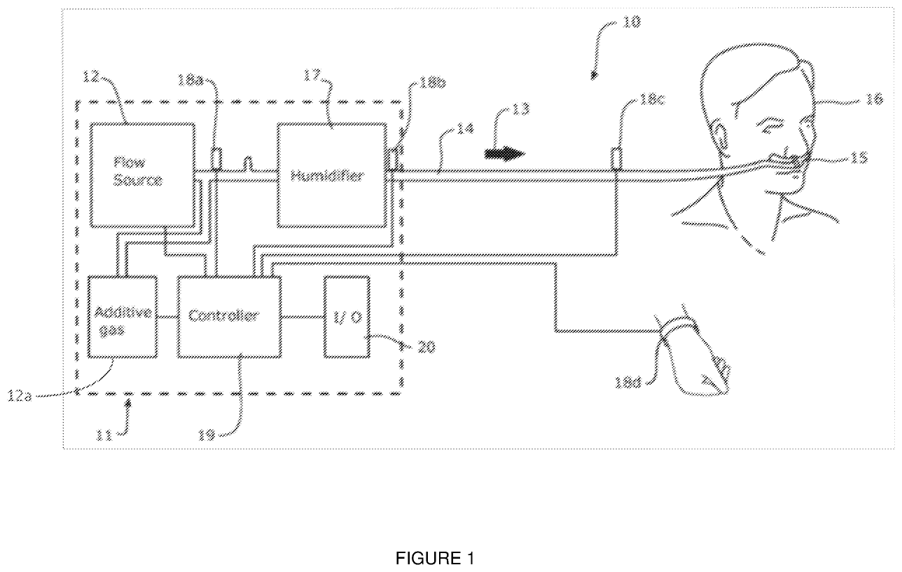

For example, a high flow respiratory system 10 is described with reference to FIG. 1. High flow may be used as a means to promote gas exchange and/or respiratory support through the provision of oxygen and/or other gases, and through the removal of carbon dioxide (CO2) from the patient's airways. High flow may be particularly useful prior to, during or after a medical and/or anaesthetic procedure.

When humidified, the high gas flow can also prevent airways from drying out, mitigating mucociliary damage, and reducing the risk of laryngospasms and risks associated with airway drying such as nose bleeding, aspiration (as a result of nose bleeding), and airway obstruction, swelling and bleeding. Another advantage of high gas flow is that the flow can clear smoke created during surgery in the air passages. For example, smoke can be created by lasers and/or cauterizing devices.

With reference to FIG. 1, the system 10 may comprise an integrated or separate component-based arrangement, generally shown in the dotted box 11 in FIG. 1. In some configurations, the system 10 could comprise a modular arrangement of components. The system 10 may include a flow source 12, such as an in-wall source of oxygen, an oxygen tank, a blower, a flow therapy apparatus, or any other source of oxygen or other gas or a combination thereof. In some embodiments, the flow source 12 comprises a flow modulator and in some embodiments, the flow modulator comprises a flow generator such as a blower, bellow, and/or pistons. In some embodiments, the flow modulator comprises a flow generator and a proportional valve which may function to control oxygen concentration in a flow of blended gas such as air as may be drawn from ambient air in the room/surroundings (preferably filtered air) and oxygen which is provided to the patient. In some embodiments, the flow modulator comprises a proportional valve, and in such embodiments, the flow modulator need not comprise a flow generator. In other embodiments, the flow source 12 need not comprise a flow generator and in such embodiments, the flow source 12 may comprise an in-wall gas source and/or a blended gas or other gas supply. In some embodiments, the flow modulator may be in pneumatic communication with a gas source, for example a blower and/or a proportional valve may be in pneumatic communication with a compressed gas source. In some embodiments, the blower and/or the proportional valve receives gases from a compressed gas source. In some embodiments, the flow source 12 may comprise a compressed gas source (e.g. an in-wall gas source, an oxygen tank supply, etc.) and a blower. In some embodiments, the system may comprise a plurality of flow modulators, such as proportional valves, each of which may be in fluid communication with a compressed gas source such that the system may receive a flow of gasses from one or more than one different gas sources. A compressed gas source may comprise an air, oxygen, nitrogen or other source of compressed medical gas.

In some embodiments, the flow source 12 comprises or is part of a respiratory apparatus such as an anaesthesia machine or a ventilator. The system 10 may also comprise an additive gas source 12a, comprising one or more other gases that can be combined with gases from the flow source 12. While FIG. 1 shows the additive gas source 12a pneumatically connected downstream of the flow source 12, it will be appreciated that the additive gas source 12a can introduce additive gases upstream, at or downstream of flow source 12. The flow source 12 can provide a flow of gas 13 that can be provided to a patient 16 via a delivery conduit 14, and patient interface 15 (such as a nasal cannula). The flow of gas 13 may provide a high flow to the patient, in the context described in the foregoing. A controller 19 controls the flow source 12 and additive gas source 12a through valves or the like to control flow and other characteristics such as any one or more of flow rate, pressure, composition, concentration, and volume of the flow of gas 13. A humidifier 17 is also optionally provided, which can humidify the gas and/or control the temperature of the gas, for example under the control of the controller 19. The flow source 12 and the humidifier 17 may together form an integrated system, or the flow source 12 and the humidifier 17 may be separate components that are pneumatically connected together. In some embodiments the flow source 12 and humidifier 17 are mechanically coupled together. In some embodiments, the humidifier 17 may be a part of a respiratory apparatus such as an anaesthesia machine or a ventilator. In embodiments where the flow of gas 13 is humidified, the system 10 is configured to provide the flow of gas 13 to a patient 16 at a temperature range of between about 27 °C and 37 °C, at a humidity of greater than about 12 mg/l. In some embodiments, the system 10 is configured to provide the flow of gas 13 to a patient at a temperature range of between about 31 °C and 37 °C. In some embodiments, the flow of gas 13 provided to a patient has a humidity of up to about 44 mg/l. One or more sensors 18a, 18b, 18c, 18d, such as flow, oxygen, pressure, humidity, temperature or other sensors can be placed throughout the system and/or at, on or near the patient 16. The sensors can include a pulse oximeter 18d on the patient for determining the oxygen concentration in the blood.

The controller 19 may be operatively coupled with one or more components of system 10 by various means including wired or wireless coupling. For example, controller 19 may be operatively coupled with one or more of the flow source 12, the additive gas source 12A, humidifier 17 and sensors 18a-18d and input/output (I/O) interface 20. By way of example, the controller 19 may be provided on or in a high flow apparatus, a separate component and/or incorporated into or utilised with another respiratory apparatus such as an anaesthesia machine or a ventilator, or it may comprise part of system 10 and communicate with one or more separate controllers controlling operation of separate components used with system 10 for the provision of respiratory support to the patient. The controller may comprise a microcontroller, a PID (proportional-integral-derivative) controller or a variation of a PID controller where the proportional, integral and derivative elements of the controller can be turned on or off as needed (such as P, PI or I controllers).

The controller 19 may comprise a processor configured to execute instructions stored in a memory device in communication with the controller to direct the operation of controllable components of the respiratory system. A storage medium may be coupled with the controller 19 such that the controller can read information from, and write information to, the storage medium. In the alternative, the storage medium may be integral to the controller and contain instructions causing the controller to operate the display screen of the interface 20 and other aspects of the system 10 in accordance with the embodiments disclosed herein. The controller 19 may further comprise circuits for receiving sensor signals.

The controller 19 may thus control the flow source 12 and other components of or used with system 10 to provide a flow of gas to the patient with certain characteristics such as a desired flow rate, pressure, composition (where more than one gas is being provided), volume and/or other parameters based on user-selections received by the controller 19 and/or feedback from one or more sensors 18a-18d. The controller 19 can also control any other suitable parameters of the flow source to meet oxygenation, airway pressure and/or flow requirements of the patient and/or system pressure and/or flow requirements of the system (for example pre-determined or set by a user through interface 20). The controller 19 may also control the humidifier 17 and this control may be based on feedback from one or more of the sensors 18a-18d. Using input from the sensors, the controller may determine operational changes required to meet oxygenation requirements and alter control parameters of the flow source 12 and/or humidifier 17 and/or other additive gas sources 12a and/or other components of the system as required. In some embodiments, the controller 19 does not control the humidifier 17.

An input/output (I/O) interface 20 comprises a display device and may comprise input/output devices such as a keypad, mouse, stylus, touch sensitive display screen or the like. The input/output (I/O) interface 20 enables information to be presented via display views made visible on the display device and inputs (such as the required patient respiratory support parameters) to be received from a user (e.g. clinician or patient). The received inputs can be used for determining oxygenation, pressure, flow requirements and/or other system settings used in the control of one or more of the flow source 12, additive gas source 12a and other components of the system 10, to achieve a flow of gas 13 with the characteristics necessary to provide the required respiratory support.

As noted above, the gases flow (optionally humidified) may be provided to the patient 16 via a delivery conduit 14 and the patient interface 15 or ‘interface’, such as a cannula, mask, nasal interface, oral device or combination thereof. In some embodiments, the gas flow (optionally humidified) may be provided to the patient 16 for surgical uses, e.g. surgical insufflation. In these embodiments, the ‘interface’ could be a surgical cannula, trocar, or other suitable interface. The patient interface may be non-sealing, substantially non-sealing, or partially non-sealing with a patient's airways. A nasal interface as used herein comprises a device such as a cannula, a nasal mask, nasal pillows, nasal prongs or other type of nasal device or combinations thereof configured to direct a flow of gas into one or both nares of the patient.

A nasal interface can also be used in combination with a face mask or oral device (such as a tube inserted into the mouth) and/or a mask or oral device (such as a tube inserted into the mouth) that can be detached and/or attached to the nasal interface.

A nasal cannula is a nasal interface that may include one or more prongs that are configured to be inserted into a patient's nasal passages. A mask refers to an interface that covers a patient's nasal passages and/or mouth and can also include devices in which portions of the mask that cover the patient's mouth are removable. A mask also refers to a nasal interface that includes nasal pillows that create a substantial seal with the patient's nostrils.

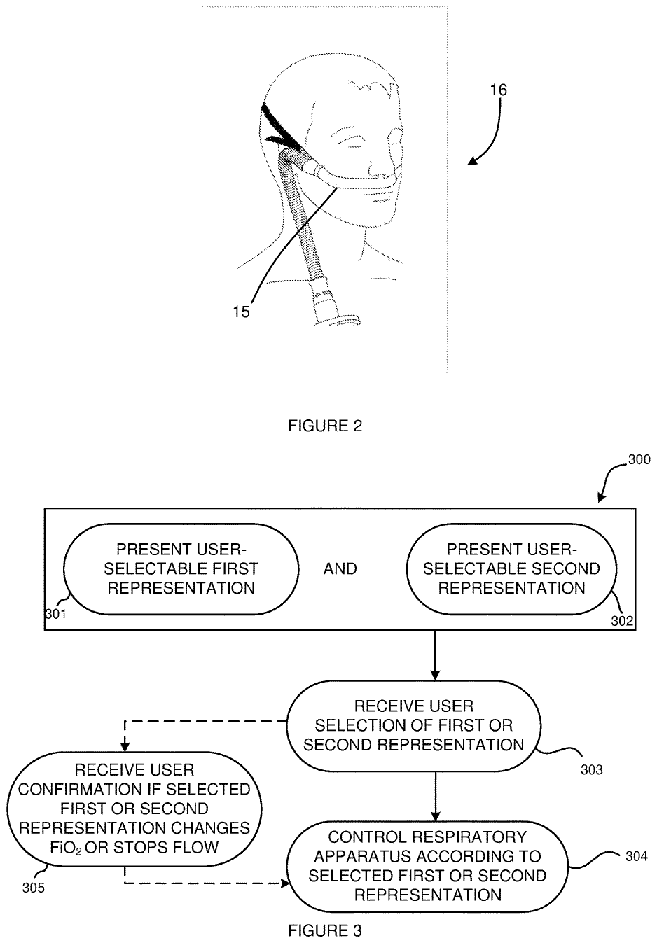

FIGS. 2, 24 and 25 shows examples of a patient 16 wearing a patient interface 15, 200 for example the nasal cannula 15 of the respiratory system 10 of FIG. 1, with a collapsible portion. The patient depicted is an adult. However, the patient may be an infant, child or adolescent.

The patient interface 200 comprises a first gas (delivery) conduit 202. The first gas conduit 202 is adapted to receive gases from the respiratory system 10 of FIG. 1 (for example, via the conduit 14 shown in FIG. 1) and direct the gases to the patient 16.

The patient interface 200 may comprise a gases delivery side arm in fluid communication with the first gas conduit 202. The first gas conduit 202 is in pneumatic communication with a flow manifold 206 which is provided at an end of the gases delivery side arm. The flow manifold 206 receives gases from the first gas conduit 202 and provides passage to one or more nasal delivery elements 208 (e.g. nasal prongs) extending from the manifold. The one or more nasal delivery elements 208 extend outwardly from the flow manifold 206.

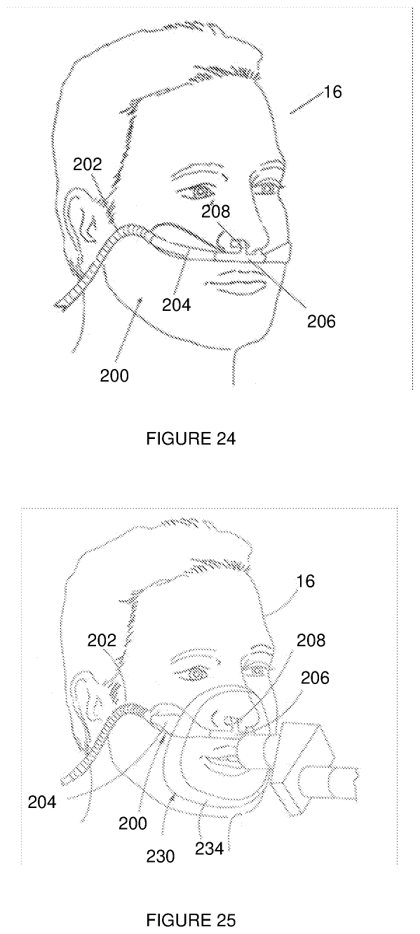

In the embodiment shown in FIGS. 24 and 25, the flow manifold 206 receives flow from one lateral side of the flow manifold 206 (e.g. with respect to an imaginary vertical plane bisecting the face of the patient 16) and provides a passage for flow through to the manifold to each of the nasal prongs 208. In some embodiments a conduit may extend from a single side of the manifold, for example from the left-hand side or from the right-hand side of the manifold.

The first gas conduit 202 of the patient interface 200 comprises a first portion 204 configured to transition from a first state in which a first level of gases is able to pass through the first portion 204 to a second state in which a second level of gases is able to pass through the first portion 204.

FIG. 25 shows the patient 16 wearing the patient interface 200 comprising two nasal prongs 208 simultaneously underneath a face mask assembly 230 (a second patient interface). In this arrangement, face mask assembly 230 is placed upon the patient interface 200 which is worn by patient 16. FIG. 25 schematically shows the face mask assembly 230 as a transparent structure in order to illustrate the patient interface 200 under it. The first patient interface 200 may be used with a first respiratory support system 10 and the face mask assembly (second patient interface) 230 may be used together with a second respiratory support system (not shown). In some examples, the first and second respiratory support systems are the same system and/or the first and second respiratory support systems comprise a common flow source despite the modes of respiratory support being provided by the first and second respiratory support systems being different. In other examples, the first and second respiratory support systems are separate systems.

The example shown in FIG. 25 may be beneficial in the provision of selective delivery of separate therapies or modes of support to a patient using different patient interfaces, and/or in stopping or ceasing the delivery of a therapy from an interface and/or allowing gases provided by an interface to be sampled. This example may find particular application in e.g. emergency resuscitation, around intubation of a patient receiving high flow therapy, ear, nose, and throat (ENT) surgery, in assisting with conditioning of a patient in a pre-operative state prior to administration of anaesthetics, and during post-extubation and recovery.

In one particular application, a patient undergoing an anaesthetic procedure may undergo pre-oxygenation by delivering a high flow of oxygen or humidified gases or mixture of both, for example via a nasal cannula, when the patient is still spontaneously breathing and before the administration of anaesthetic agents. Pre-oxygenation increases the patient's oxygen reservoir prior to the anaesthetic procedure. The term “anaesthetic procedure” may refer, without limitation, to general anaesthesia, procedural sedation and regional/local anaesthesia. In some circumstances, anaesthetists managing the anaesthetic procedure of a patient may want to switch between delivery of gas flow from one patient interface (for example a nasal cannula 200) and delivery of gas flow from another patient interface, such as via a face mask 230.

Anaesthetists may also use a mask with a bag to oxygenate a patient, and in some instances find it more beneficial to use a bag mask if a patient's vital signs begin to drop for example to deliver more pressure to support the patient's airways, or to have greater manual control over the variation in delivered pressure. In some situations, a medical professional may wish to switch between different respiratory systems or support modes. In a first mode, respiratory support may be provided by a first respiratory support system (for example via the patient interface 200) and in a second mode respiratory support may be provided by a second respiratory support system (for example via the face mask assembly 230), with the support from the first system reduced or stopped. For example, it may be desirable to stop flow from a first patient interface 200 when delivering anaesthetic agents through the face mask assembly 230 because a flow from interface 200 may modify the expected behaviour of the anaesthetic circuit provided by the face mask 230 (which is typically a sealed circuit) and may dilute anaesthetic agents delivered by face mask assembly 230. Thus, it may be advantageous to be able to stop the additional flow from the first respiratory system or substantially reduce it.

In some configurations, the switching between two respiratory support modes or subsystems may be facilitated by a structure of the first gas conduit 202, which has first portion 204 configured to transition between a first state in which a first level of gases is able to pass through the first portion 204 and a second state in which a second level of gases is able to pass through the first portion 204.

In some embodiments, the first portion 204 is configured to be more collapsible or otherwise better adapted for changing the flow of gas through the first portion 204 (to reduce the flow of gas through the conduit and to the patient) than other portions of the conduit 202, and/or allowing a seal of a mask to seal over the top of the conduit. In other embodiments the entire conduit 202 may be configured to be collapsible or otherwise better adapted for changing the flow of gas through conduit 202.

In some embodiments, the first state is a fully or substantially open condition, and the second state is a fully or substantially closed condition. That is, the conduit 202 is configured to be more collapsible, deformable or otherwise adapted to fully or substantially close off the flow at the first portion 204 than at other portions of the conduit 202, when in the second state. It will be understood that there may be one or more intermediate states between the first state and second state, where these one or more intermediate states may be less open (or more closed) than the fully or substantially open state (first state) but more open (or less closed) than the fully or substantially closed state (second state).

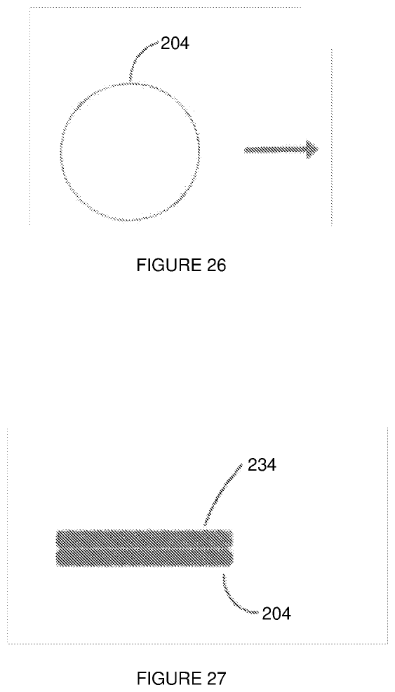

FIGS. 26 and 27 are schematic illustrations of the first state and second state. FIG. 26 shows the first portion 204 in the first state, that is a substantially open state and FIG. 27 shows the first portion 204 in the second state, that is a substantially closed state by application of the seal 234 of face mask 230 over the first portion 204. In some embodiments, the first portion 204 i.e. the more collapsible or deformable section of the first gas conduit 202 should be of a length that is greater than a width of a section of a seal 234 of the face mask 230 that bears over the first portion 204 of the first gas conduit 202. This ensures the seal of the face mask 230 does not bear over a non-collapsible section of the first gas conduit 202.

Switching between respiratory support therapies may therefore be achieved simply by applying a mask to the patient's face so that the seal of the mask partially or completely collapses the first portion 204 of the gas conduit 202 supplying the first interface 200 to reduce or stop the therapy supplied by the first interface 200. This also provides a seal between the face mask 230 and the external surface of the first portion 204 of the conduit 202 such that respiratory support or therapy can be provided by the face mask 230 where the respiratory support or therapy provided by the first patient interface 200 can be reduced or shut off. The patient interface 200 with a collapsible conduit portion 204 allows a user, e.g. an anaesthetist or a nurse or a clinician, to use a face mask assembly 230 over the patient interface 200 to select and control delivery of gases from multiple respiratory support systems to provide different therapies or modes of support. The first patient interface 200 may be structured to function in a manner that prevents the delivery of flow and other respiratory therapy or anaesthetic agents through the patient interface 200 when the first portion 204 is in a second state. In some embodiments removal of the face mask assembly 230 from the patient's face allows the first portion 204 to return to its first state so that respiratory support or therapy supplied by the first patient interface 200 can recommence or return to operating in the conditions present prior to the change in state.

FIG. 3 shows a flow chart representing a method 300 for operating a respiratory apparatus which may comprise a flow source 12, comprising the steps of presenting on a display screen a display view comprising a user-selectable first representation of a flow parameter in step 301 and a user-selectable second representation being a flow parameter set point, in step 302. The first representation enables the selection of the flow parameter value from a range of flow parameter values whereas the second representation comprises a single flow parameter set point value that may be selected by the user. The display view comprises a visible output on a display screen of a display device which may comprise part of the I/O interface 20 as exemplified in FIG. 1. The display device may comprise part of a respiratory apparatus comprising a high flow device. A high flow device may comprise a flow source configured to provide high flow respiratory support and may optionally include one or more of a controller, humidifier, proportional valves and sensors for providing the required respiratory support. Alternatively or additionally, the display device may comprise part of, or be used in the control of, an anaesthesia machine or ventilator which is separate from and used in the control of a high flow device. Communication between the anaesthesia machine or ventilator and the high flow device may be via a physical communication link and/or a wireless link utilising transceivers and/or communication protocols implemented in the respective components. In some embodiments, the display device comprises part of an anaesthesia machine or ventilator that is itself, capable of providing high flow respiratory gases. In other embodiments, the display device may comprise part of a high flow device that is configured to couple with an anaesthesia machine or ventilator. The coupling may comprise a physical interface in the form of a dock although that need not be the case and a communicative coupling without physical connection may be provided. A dock, when provided, may or may not comprise a recess into which the high flow device is inserted. The high flow device may be functionally couplable with the anaesthesia machine or ventilator to add functionality that permits provision of high flow respiratory support in addition to anaesthesia/ventilation, and may be detachable for provision of high flow support separately from the anaesthesia machine/ventilator. In some embodiments, the display device does not form part of the device providing the high flow of respiratory gases, but is in operative communication with it.

In some embodiments, the first representation and the second representation are presented on the display view simultaneously, or substantially simultaneously, and/or appear contemporaneously on the display view. This may occur following start-up of the device such that presentation of the first representation and the second representation are the first operational features presented on the display view after powering up the device. In a step 303, a user selection of the second representation is received, and, responsive to the user selection, the respiratory apparatus is controlled in step 304 to provide a gases flow according to the parameter set point value corresponding to the second representation. Additionally and/or alternatively in a step 303, a user has the option of making a selection of the first representation which may be received and, responsive to that user selection, the respiratory apparatus is controlled in a step 304 to provide a gases flow according to the user's selected value from the range corresponding to the first representation.

In some embodiments, the display view which may or may not comprise part of the respiratory apparatus providing the flow of gas, is controlled by or is in operative communication with a controller 19 as exemplified in FIG. 1. Thus, the user selections of the representations presented on the display view are received by the controller 19 which, responsive to the user selections, causes control of the respiratory apparatus according to the user selection. Advantageously, presentation of the first representation and second representation can permit a user to quickly and easily make the selections required to provide respiratory support to the patient in a ‘one-step’ selection. In some embodiments, when the user makes a selection using the first representation or the second representation, no confirmation input is required since the respiratory apparatus may be configured on start-up to provide gases at 21% FiO2 at least, and up to 100% FiO2 and at OLPM. There is no requirement for the user to navigate through menu selections or progress through multiple display views before respiratory support can be commenced. Confirmation may be required by the controller in an optional step 305 (designated by broken lines) if the user makes a selection that involves changing FiO2 and/or stopping flow. The respiratory apparatus may comprise a flow source such as at least one of a blower and a valve such that the method 300 comprises controlling the blower and/or the valve to provide the gases flow according to the parameter value corresponding to the first representation or the second representation. The gases flow may be provided to a patient via a non-sealing interface, such as a nasal cannula as shown in FIG. 2, that is provided in fluid communication with a gases outlet of the respiratory apparatus.

In some embodiments, the display screen comprises a touch screen on a display device which enables the user to make selections of the user-selectable representations by touching those representations on the display view. However it is to be understood that this need not be the case, and that other input devices may be provided as part of the I/O interface 20 that facilitate user-selection of the first or second representations (or third representation, as discussed below) using other means such as a mouse, keypad, stylus, pointer or the like.

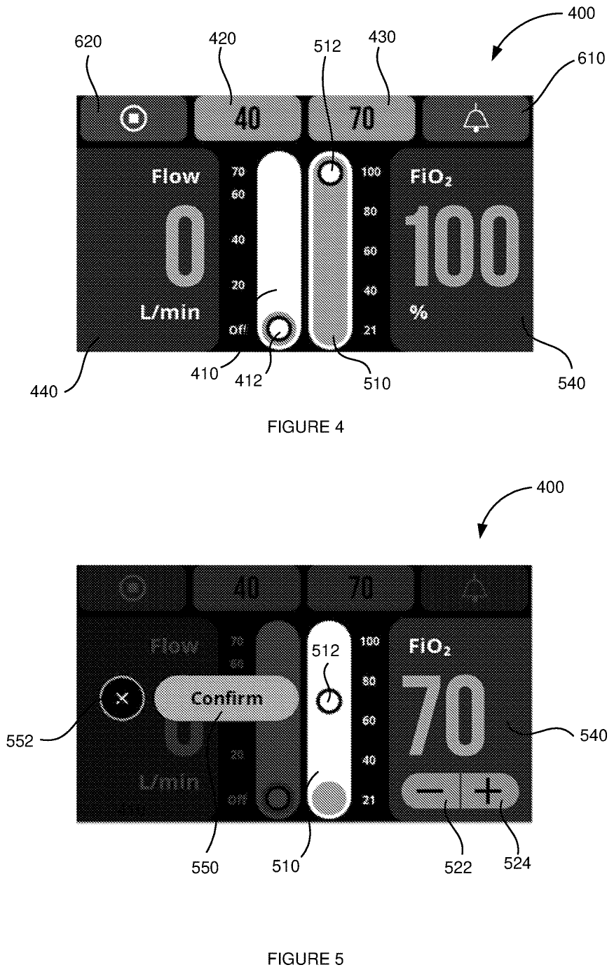

Methods of controlling the respiratory apparatus will be explained in further detail by reference to examples of display views presented on a display device of interface 20 as shown in FIGS. 4 to 7 and 9 to 21.

FIG. 4 illustrates a display view 400 presented on a display screen and used in controlling a respiratory apparatus which provides for quick selection of a flow parameter for provision of respiratory support to a patient. In the embodiments shown, the user-selectable first representation 410 and the user-selectable second representation 420 may be discrete representations presented substantially simultaneously on a display view of the display device. A user-selectable third representation 430, which may be discrete from the first representation 410 and second representation 420 may also be provided. The third representation 430 may be presented on the display view simultaneously, or substantially simultaneously, and/or appear contemporaneously on the display view with the first representation 410 and/or the second representation 420.

In the embodiment shown, user-selectable first representation 410 comprises a slider that enables selection of a value from a range of available values for a flow parameter. The flow parameter may be flow rate and the user-selectable first representation 410 may present a range of values from e.g. 0 LPM to e.g. 70 LPM which may be selectable by the user by tapping at the desired value on the slider, or dragging the control pointer 412 along the slider to the desired value. The values on the slider may be selectable in increments of e.g. 1, 2, 5, 10 or 20 LPM. Flow rates of at least about 15 LPM may be provided. In some embodiments, the user-selectable first representation may provide a minimum value greater than the lowest possible value, for example the minimum available flow rate value may be 15 LPM and not 0 LPM. The increments between values may be consistent or inconsistent (for example in FIG. 4 where the values are shown in increments of 20 and 10 LPM). The values between the shown increments may be pre-set or configurable by the user. In some embodiments, the slider background may be in a contrasting colour (e.g. white) relative to the majority of the rest of the interface (darker colour).

While first representation 410 is shown as a slider in the example provided, it is to be understood that alternative or additional representations for the range of selectable values may comprise a dial, up/down arrows; +/− symbols; and/or other symbols representing an increase or decrease to the flow parameter value within the available range. For example, the display view 400 may include an additional representation such as a user-selectable representation in the form of +/− buttons as shown in FIG. 21, where the first representation 410 provides a coarse control of a user-selected value and the additional user-selectable representation provides a fine (or finer than the first representation 410) control of the user-selected value, or vice versa. The control pointer 412 may contain a coloured element, such as around the periphery of the pointer, which is coloured with a colour that may be used consistently with other representations of the same flow parameter, e.g. flow rate. In the embodiment shown, the control pointer 412 may comprise a blue outline which is coloured consistently with other elements of the display view that represent the same flow parameter, such as the second and third representations 420, 430 which also represent flow rate values. When the slider is moved or a non-zero value is selected for the first representation 410, part of the slider between zero and the pointer 412 may be filled with colour to assist with identifying the value selected. Thus, when a user selects the maximum value of the first representation, the slider portion coloured white in FIG. 4 becomes coloured e.g. blue.

In contrast to the user-selectable first representation 410, user-selectable second representation 420 comprises a single set point value which in the embodiment shown, is provided as “40” representing 40 LPM. It is to be understood that while the units of the flow parameter are not shown in the display view of FIG. 4, those units may be included in the first and/or second and/or third representations 410, 420, 430. In some embodiments, the display view 400 may provide a user-selectable third representation 430 which comprises a single set point value which in the embodiment shown, is provided as “70” representing 70 LPM. Upon selection of the second representation 420, the respiratory apparatus is controlled, e.g. by controller 19 which is in operative communication with the user interface and receiving the user selection, to provide the flow of gases according to the selected flow parameter which, according to the example shown, is 40 LPM. Upon selection of the third representation 430 (if provided), the respiratory apparatus is controlled in a similar fashion, to provide the flow of gases according to the selected flow parameter which, according to the example shown, is 70 LPM. It is to be understood however that this is one example only and the second representation 420 and/or the third representation 430 may be programmed in a memory device containing instructions for the controller 19, to represent a different pre-set flow parameter value. For example the second representation may represent the value 70 LPM and the third representation 430 may represent the value 40 LPM effectively swapping the selectable set point values from FIG. 4. Other single numeric values may be represented in the second and third representations 420, 430. For example, other single numeric values represented in the first and second representations 420, 430 may comprise e.g. 10, 20, 30, 40, 50, 60, 70, 80, 90 or 100 LPM or numeric values between those values such as e.g. 5, 10, 15, . . . 90, 95, 100 LPM or 2, 4, 6, 8, 10, . . . 90, 92, 94, 96, 98, 100 LPM. In some examples, the single numeric values represented in the first and second representations 420, 430, are configurable by a user who may prefer particular flow parameters for “quick selection”. Such flow parameters may comprise e.g. flow rate and/or FiO2. Configuration may be achieved by the user making user preference selections using the I/O interface 20. In some embodiments, upon start-up the controller 19 causes the display device to present the quick select keys according to their most recently saved configuration. In some examples, the user may provide via the I/O device 20 an identification input that enables the controller to retrieve pre-configured user preference selections for that user.

Advantageously, provision of a user-selectable single set point value of the flow parameter in the second and/or third representations 420, 430 enables quick selection of the flow parameter value for provision of e.g. high flow therapy to the patient. In some embodiments, these quick select keys or “representations” 420, 430 enable a user, such as a clinician, to switch between desired used flow rates. In particular, these quick select keys or “representations” 420, 430 enable a user, such as a clinician, to switch between desired flow rates (or any other flow parameter) within a respiratory support mode (e.g. high flow mode). Switching between these flow rates (or any other flow parameter) does not change the respiratory support mode of the respiratory apparatus. Desired flow rates used during a preoxygenation stage of an anaesthetic procedure may comprise e.g. 40 LPM at a range of about 21% to about 100% FiO2 (Fraction of Inspired Oxygen) while the patient is spontaneously breathing (i.e. pre-oxygenation phase), for example, 40 LPM at 100% FiO2.

After an anaesthetic agent has been administered and/or when the patient is apnoeic (i.e. apnoeic phase), or has diminished or is at risk of diminished respiratory function, desired flow rates may comprise e.g. 70 LPM and a range of about 21% to about 100% FiO2, for example 70 LPM and 100% FiO2. Quick selection of these flow rate set points using a single interaction with the I/O interface 20 is made possible according to the present disclosure, while other flow parameters, such as FiO2 remain unchanged. This enables the clinician to quickly and simply switch between the two flow rate set points using the quick select keys comprising the second and third representations 420, 430. Quick select keys may simplify operation and control of a respiratory apparatus providing respiratory support to a patient, by avoiding multiple view and/or menu and/or parameter selections, and/or minimizing inaccurate or erroneous selection of flow parameters. This may be particularly beneficial if respiratory apparatus is receiving a flow of gases at FiO2 of 100%, since quick select keys permit the flow rate to be set to the required level with a single selection, and in the case of embodiments where the display device comprises a touch screen, with a single touch. In some embodiments, there may be several quick select keys that permit a user to change the value of one, or of more than one flow parameters with a single selection. In some examples, the quick select keys may be located substantially centred in a horizontal and/or vertical aspect of the display view, or in another preferred location for ease of selection by the user. In some embodiments, the quick select keys may be represented in a manner that provides greater contrast against other features presented on the display view. This may involve one or more quick select keys being presented in a contrasting colour, texture, size, typeface, or with a dynamic feature such as flashing, scrolling or other animation that differs from surrounding elements of the display view.

In some embodiments, one or more quick select keys may present to the user a quantitative e.g. numerical value and/or units corresponding to the flow parameter value that can be selected. Alternatively, or additionally one or more quick select keys may comprise a label or text that provides a qualitative indicator of the flow parameter value that can be selected. The qualitative indicator may correspond to e.g. a mode of respiratory support to be provided. For example, FIRST FLOW for a flow rate value corresponding to 40 LPM which may correspond to the second representation 420. In other examples, the qualitative indicator may correspond to e.g. a breathing descriptor or phase of induction. For example, PREOX or SPONT or AWAKE corresponding to a pre-oxygenation phase during which the flow rate may be controlled according to the second representation 420 which may control the respiratory apparatus to a preset value e.g. 40 LPM. In another example, SECOND FLOW may be a qualitative indicator for a flow rate value corresponding to 70 LPM which may correspond to the third representation 430. Other qualitative indicators may be associated with a breathing descriptor or phase of induction occurring after anaesthetic agent has been administered and/or when the patient is apnoeic, or has diminished or is at risk of diminished respiratory function. For example, APNEA, GA (for General Anaesthetic), SEDATION, ASLEEP, PROCEDURE corresponding to a period during which the flow rate value may be controlled according to the third representation 430 which may control the respiratory apparatus to a preset value e.g. 70 LPM. As noted above, the pre-set values corresponding to the first and second representations (and third representation, when provided) may be customisable by the user. In some embodiments, the selection of a qualitative indicator may also change the value of a further pre-set parameter. In one non-limiting example, selection of APNEA may change selection of the value of flow rate to 70LPM and FiO2 to 100%. In another non-limiting example, selection of PREOX may change selection of the value of flow rate to 40 LPM and FiO2 to 100%. This may or may not require confirmation input from the user. In some embodiments, the respiratory apparatus may be configured such that upon start-up, the controller controls the flow source to provide a flow of gases corresponding to a PREOX setting e.g. corresponding to a flow rate value of 40 LPM and about 100% FiO2. This enables flow to be delivered almost immediately upon start-up of the apparatus.

Throughout this disclosure, the term FiO2 is used as a convenient label for the oxygen (O2) content of gases provided by the respiratory apparatus. Thus, FiO2 as used herein designates the concentration of O2 in the flow of gases although the traditional definition of FiO2 is the Fraction of Inspired Oxygen in gases inhaled by the patient. In some examples, the O2 content of the gases inhaled by the patient may be the same as the O2 content of gases provided by the respiratory apparatus. However, that may not always be the case due to e.g. leaks in the system, entrainment by the patient and/or the patient's inhalation efforts. Thus, it is to be understood that the term FiO2 as used herein broadly refers to the O2 content provided by the respiratory apparatus which may be the same as or different to the actual FiO2 of the patient. In some contexts, the O2 content of gases provided by the respiratory apparatus may be referred to as a Fraction of Delivered Oxygen (FdO2).

In some embodiments, if the respiratory apparatus has an O2 supply connected and entering the respiratory apparatus, the device will default to, upon start-up, operating at 0 LPM and 100% FiO2. That is, upon startup, the controller may set a flow parameter for the gases flow to a predetermined oxygen concentration value which may, in some examples, be 100% FiO2. In other examples, the predetermined oxygen concentration value may be e.g. 21% FiO2 or some other value. The desired flow rate can then be selected by user selection of a flow rate set point using the quick select keys (second representation 420 or the third representation 430), or by user selection of a flow rate from the range of available values using the first representation 410. Upon selection of the flow rate, the controller controls the respiratory apparatus to provide the gases flow at the predetermined oxygen concentration value and the user-selected flow rate.

Advantageously, when quick select keys are presented on a commonly used display view such as a main view presented on the display screen the user can immediately see and select the required or desired flow rate set point using the second representation 420, and optionally the third representation 430, thereby controlling operation of the respiratory apparatus to provide flows at the selected parameter set point value. A main view may comprise the first interactive display view presented after power up and device initialisation is complete. Upon presentation of the main view the user can immediately commence therapy providing flow at the rate corresponding to the set point value shown by second representation 420 (or the third representation 430 where provided).

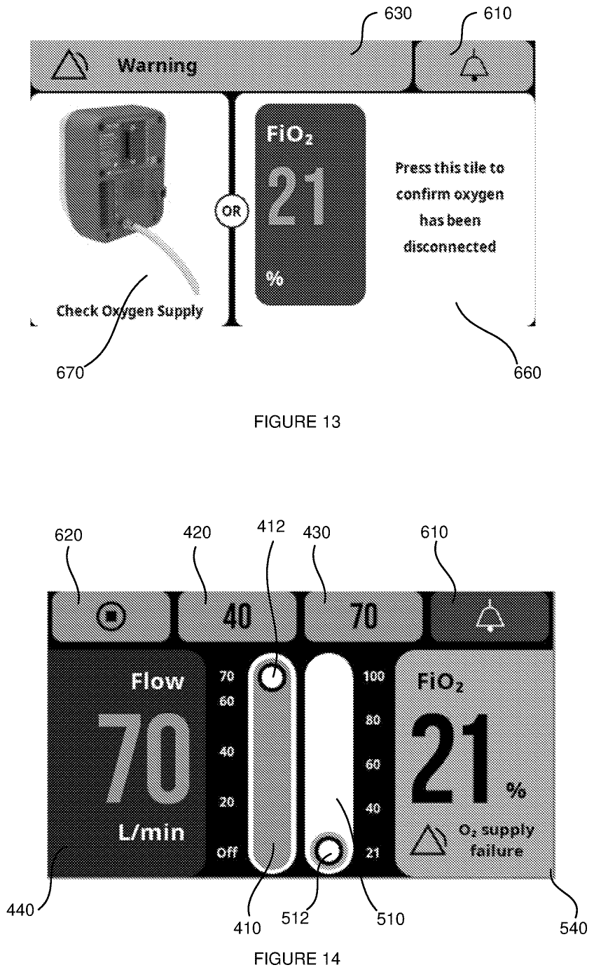

In some embodiments, if there is no O2 supply entering the respiratory apparatus, the device will default to, upon start-up, operating at OLPM flow and 21% FiO2 which is representative of the O2 concentration of ambient air. An alert and/or a warning and/or user guidance may be presented on the display view if the user attempts to change FiO2 to higher or lower than 21% as discussed below in relation to FIGS. 12 and 13.

Presentation on the display view of the first representation 410 still permits selection of other flow parameter values within a displayed range for cases where the quick select values are not the flow parameter values that the clinician wishes to use for a given patient and/or procedure. Additionally, the display view 400 provides an indication of the selected flow parameter value in region 440. Ideally the representation of the selected value in region 440 is relatively larger than the quick select values in the second and third representations 420, 430, and larger than the first representation 410, so that a clinician may see at a glance the selected flow parameter setting (e.g. flow rate) that has been selected for operation of the respiratory apparatus. The colour of the displayed value in region 440 may correspond with the other flow parameter values of that type presented on the display view (e.g. blue).

In some embodiments, display view 400 also provides an indication of a different flow parameter from the flow parameter corresponding to the first, second and third representations 410, 420, 430. In the example of FIG. 4, a user-selectable further representation 510 which is discrete from the first, second and third representations 410, 420, 430 may be presented on the display view simultaneously or substantially simultaneously with, and/or appear contemporaneously on the display view with the first, second and third representations 410, 420, 430. The different flow parameter may be e.g. FiO2 and the further selectable representation 510 may present a range of values from e.g. 21% FiO2 (corresponding to most room air) to 100% FiO2 which may be selectable by the user by tapping at the desired value on the slider, or dragging the control pointer 512 along the slider to the desired value. The values on the slider may be selectable in increments of e.g. 1, 2, 5, 10 or 20%. In some embodiments, alternative FiO2 units may be used, e.g. decimals where 1 is equivalent to 100% and 0.21 is equivalent to 21%. In the embodiment shown, the values are shown in increments of 20% but values between the shown values may be selected by the user. In some embodiments, the slider background may be in a contrasting colour (e.g. white) relative to the majority of the rest of the interface (darker colour). While further representation 510 is shown as a slider in the example provided, it is to be understood that alternative or additional representations for the range of selectable values may comprise a dial, up/down arrows; +/− symbols (items 522, 524 in FIG. 21); and/or other symbols representing an increase or decrease to the flow parameter value within the available range. Further representation 510 can therefore provide a coarse control of a user-selected value and the alternative or additional representations 522, 524 can provide a fine (or finer than the further representation 510) control of the user-selected value, or vice versa. The control pointer 512 may contain a coloured element, such as around the periphery of the pointer, which is coloured with a colour (e.g. green) that may be used consistently with other representations of the same flow parameter, e.g. FiO2. In the embodiment shown, the control pointer 512 may comprise a green outline which is coloured consistently with other elements of the display view that represent the same flow parameter. When the slider is moved or a non-zero value is selected for the further representation 510, part of the slider between zero and the pointer 512 fills with colour (e.g. green) to assist with identifying the selected value. Thus, when a user selects the maximum value of the further representation 510, the portion of the slider coloured white in FIG. 4 becomes coloured e.g. green.

In some embodiments, the first representation 410 and/or the further representation 510 may be located substantially centrally of the display view. When these are represented as a slider, the slider may be oriented vertically as shown, or horizontally or in other orientations. Where multiple sliders or dials are provided, e.g. corresponding to first representation 410 and further representation 510, they may be located in the display view adjacent to one another, or in close proximity.

In some embodiments, the further flow parameter may be set using a different selectable representation than further representation 510. This may be as an alternative to or in addition to the further representation 510. The example of FIG. 5 provides a user-selectable representation in the form of +/− buttons 522, 524 that permit finer control of the user-selected value of e.g. the FiO2 setting than slider 510.

In some embodiments, the display view 400 may provide an indication of the selected flow parameter value, e.g. selected FiO2 value, in region 540. The representation of the selected value in region 540 may be relatively larger than the ranges of values presented in the further representations 510 (and/or 522, 524) so that a clinician may see at a glance the selected flow parameter setting (e.g. FiO2) that has been selected for operation of the respiratory apparatus. The colour of the displayed value in region 540 may correspond with the other flow parameter values of that type presented on the display view (e.g. green).

It may be desirable, or a requirement under manufacturer or regulatory standards, for one or more user selections of FiO2 to be confirmed by the user prior to the received selection being used by the controller to control the respiratory apparatus to that selected setting. For example, if the selected FiO2 value is less than 100% it may be a requirement to obtain confirmation from the user that this is intended. Therefore, in some embodiments the method of controlling the respiratory apparatus may comprise the step of presenting a confirmation prompt 550 as shown in FIG. 5. In some embodiments, the components of the display view sitting behind the confirmation prompt 550 are relatively dimmed while the confirmation prompt is presented. The confirmation prompt may be presented over representations of the display view that are not being altered by the user's current selection. In some embodiments, the confirmation prompt 550 and/or cross 552 may move with the control pointer 512 to remain adjacent to it as the user slides or selects a new FiO2 value. User selection of the confirmation prompt 550 confirms the selection of, in the example shown, a FiO2 value of 70%, and the confirmed user selection is then used to control the respiratory apparatus. If the user does not wish to confirm the selection, they can instead select the cross 552 which removes the confirmation prompt 550 and cross 552 and returns to the previous display view without changing the control to the respiratory apparatus. In some embodiments, if the user does not select the confirmation prompt 550 within a pre-defined time (e.g. 10 sec), the confirmation prompt 550 and cross 552 disappear and the previous display view is presented without changing the control to the respiratory apparatus. In some embodiments, the confirmation prompt 550 is provided on another display view 400.

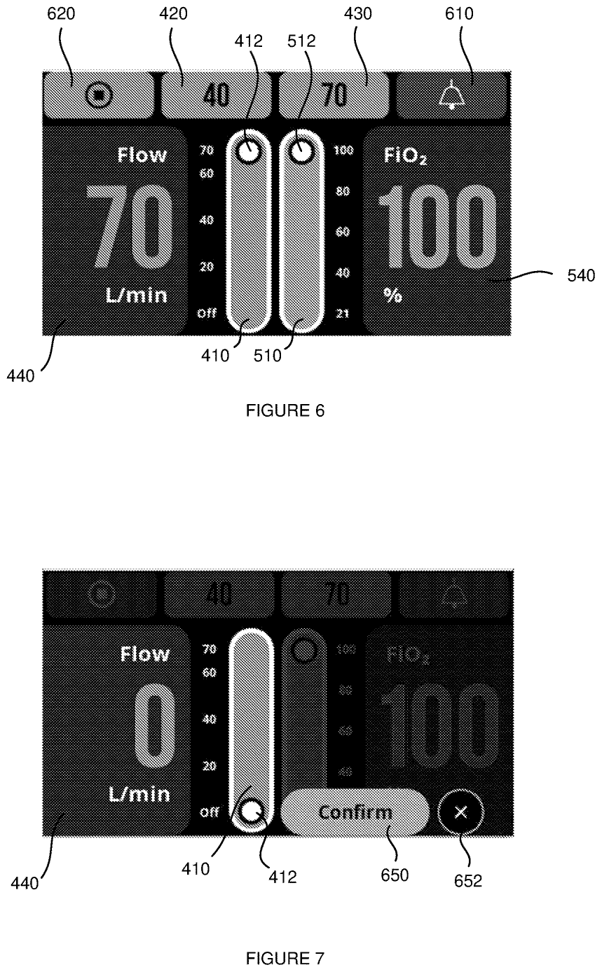

FIG. 6 illustrates a display view of the disclosure when a flow rate of 70 LPM has been selected (either using quick select key 430 or slider pointer 412) and FiO2 of 100%. In some embodiments, the default control of FiO2 may comprise a 100% setting such that no confirmation of a lower FiO2 value is required to commence therapy, once the required flow rate has been set, e.g. using the quick select keys 420, 430. An alarm element 610 may be provided on the display view which, in some embodiments, changes colour and/or flashes and/or is associated with an audible cue from the interface 20 to alert the user to an error condition in the respiratory apparatus as discussed below. In some examples, alarm element 610 may be selectable by the user which upon selection, silences the audible cue for a predetermined period (e.g. 10 or 20 seconds). If the error condition is not resolved during the silenced period, the audible cue will be presented again. Additionally, a user-selectable stop element 620 may be provided on the display view which may be selected by the user to stop the therapy.

If a user selection is received to stop therapy, e.g. by selection of stop element 620 to stop flow, the method of controlling the respiratory apparatus may comprise the step of presenting a confirmation prompt 650 as shown in FIG. 7. In some embodiments, the components of the display view sitting behind the confirmation prompt 650 are relatively dimmed while the confirmation prompt is presented. The confirmation prompt may be presented over representations of the display view that are not being altered by the user's current selection. Since a user selection to stop therapy involves reducing flow rate to zero, the display view may present the confirmation prompt 650 over dimmed elements of the display view relating to FiO2, so that the user can confirm selection of a flow rate of 0 LPM. In some embodiments, the confirmation prompt 650 and/or cross 652 may move with the control pointer 612 to remain adjacent to it as the user slides or selects a 0 LPM flow rate. User selection of the confirmation prompt 650 confirms the selection of 0 LPM which is then used to control the respiratory apparatus to stop flow to the gases outlet. If the user does not wish to confirm the selection, they can instead select the cross 652 which removes the confirmation prompt 650 and cross 652 and returns to the previous display view without changing the control to the respiratory apparatus. In some embodiments, if the user does not select the confirmation prompt 650 within a pre-defined time (e.g. 10 sec), the confirmation prompt 650 and cross 652 disappear and the previous display view may be presented without stopping flow in the respiratory apparatus. In some embodiments, the confirmation prompt 550 is provided on another display view 400. In some embodiments, the confirmation prompt may be used to seek confirmation of user selections of different flow rates however this may not be necessary or desirable if not dictated by manufacturer or regulatory standards since confirmation of flow rate selections adds a further step to the selection process which can add delay to the provision of therapy.

In some embodiments, the user-selectable stop element 620 may be provided in close proximity to one or more of the user-selectable first, second and third representations 410, 420, 430 since all relate to control of flow rate of gases from the respiratory apparatus. The stop element 620 may also be represented with a colour element that is consistent with other elements or representations on the display view that relate to flow rate, such as one or more of the first, second and third representations 410, 420, 430 and the displayed value at 440. In some embodiments, the stop element 620 may be represented with a colour element that changes depending on operation of the respiratory apparatus. For example, when the respiratory apparatus is not providing flow the stop element 620 may be represented in a different or contrasting colour compared to the representation of the value in region 440, or the stop element may be represented in the background colour of region 440 making it more visually apparent to the user that the respiratory apparatus is not providing flow. In another example, when the respiratory apparatus is providing flow, the stop element 620 may be represented in a colour that corresponds to the colour of the value in region 440 making it more visually apparent to the user that the respiratory apparatus is providing flow, and that flow may be stopped by selection of the stop element 620.

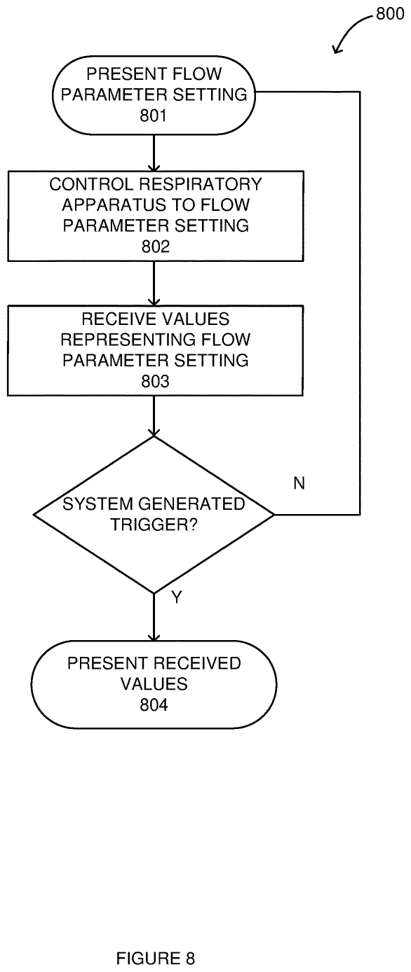

Another aspect of the disclosure relates to presenting on a display screen a display view used in controlling a respiratory apparatus which provides for automatic switching of the display view content when a system-generated trigger condition is met. FIG. 8 is a flow chart representing a method 800 for operating a respiratory apparatus which may comprise a flow source 12, comprising the steps of presenting on a display screen a display view comprising a representation of a flow parameter setting in a step 801, controlling the respiratory apparatus to provide the gases flow according to the flow parameter setting in a step 802, receiving values representing a flow parameter of the gases flow provided by the respiratory apparatus in a step 803 and responsive to receiving a system-generated trigger, automatically causing the display view in a step 804, to replace the representation of the flow parameter setting with presentation of the received values representing the flow parameter of the gases flow. It is to be understood that a system-generated trigger is one that may be triggered by a device, controller, processor, sensor or other element of the respiratory apparatus, or components outside the respiratory apparatus but forming part of the overall system providing respiratory support to the patient, as opposed to a trigger that is user-generated. System-generated triggers may comprise ones that are triggered by an instrumented (e.g. non-human initiated) input. In some examples, an instrumented input may represent one or more of a condition, state, observation or performance measure of an aspect of the respiratory apparatus and/or the patient. In some examples, system-generated triggers do not comprise time-based triggers solely activated by a clock, timer, time delay or time-based measure in the absence of some other system-generated non-time based input.

In some embodiments, the display view is controlled by a controller associated with or comprising part of a controller 19 as exemplified in FIG. 1. The display view comprises a visible output on a display screen of a display device such as I/O interface 20. The display device may comprise part of a respiratory apparatus comprising a high flow device. A high flow device may comprise a flow source configured to provide high flow respiratory support and may optionally include one or more of a controller, humidifier, proportional valves and sensors for providing the required respiratory support. Alternatively or additionally, the display device may comprise part of, or be used in the control of, an anaesthesia machine or ventilator which is separate from and used in the control of a high flow device. Communication between the anaesthesia machine or ventilator and the high flow device may be via a physical communication link and/or a wireless link utilising transceivers and/or communication protocols implemented in the respective components. In some embodiments, the display device comprises part of an anaesthesia machine or ventilator that is itself, capable of providing high flow respiratory gases. In other embodiments, the display device may comprise part of a high flow device that is configured to couple with an anaesthesia machine or ventilator. The coupling may comprise a physical interface in the form of a dock although that need not be the case and a communicative coupling without physical connection may be provided. A dock, when provided, may or may not comprise a recess into which the high flow device is inserted. The high flow device may be functionally couplable with the anaesthesia machine or ventilator to add functionality that permits provision of high flow respiratory support in addition to anaesthesia/ventilation, and may be detachable for provision of high flow support separately from the anaesthesia machine/ventilator. In some embodiments, the display device does not form part of the device providing the high flow of respiratory gases, but is in operative communication with it.

One or more flow parameter settings are presented on the display view during normal operation of the respiratory apparatus. The one or more flow parameter settings may have been selected by the user defining a “set point” e.g. by selection of representations 410, 420, 430, 510, 522, 524 for flow rate and/or FiO2. The respiratory apparatus is controlled to provide a flow of gas according to the one or more displayed flow parameter settings, or set points, as shown on the display view. Meanwhile, values representing a flow parameter of the gases flow provided by the respiratory apparatus are received. When a system-generated trigger is received by the controller, the representation of the selected flow parameter (set point) presented on the display view is automatically replaced with presentation of the received values representing the flow parameter of the gases flow.

Receiving a system generated trigger may comprise the controller determining e.g. a mis-match between received values representing the flow parameter and a pre-determined relationship which may be stored in a memory device associated with the controller, and/or a physiological indicator stored in a memory device associated with the controller being reached, and/or detection of a breathing interface providing a gases flow to a patient, or that a threshold has been crossed, as will be discussed below. In order for the controller to determine if a system trigger has been received, the controller may receive signals from one or more sensors or other hardware components of the respiratory system as will be discussed below.

Existing ventilators and other respiratory apparatuses typically show the characteristics of flow provided to the patient over time in a graphical format, and may elicit an audible and/or visible alert when set points are not met. However, aside from the existence of the alarm condition, it can be difficult for the user to determine at a glance the characteristics of the actual flow that is being provided to the patient (or to a patient interface) once the alarm condition has been met. For example, if a flow rate set point is not met, it can be difficult to ascertain from the graphical representation of flow over time, the flow rate being provided to the patient (or to the patient interface) after the alarm condition has been met. Alternatively or additionally, if a FiO2 set point is not met, it can be difficult to ascertain from the graphical representation the FiO2 percentage being provided after the alarm condition has been met.

By controlling the display controller to present the received values of the flow parameter instead of or in addition to the set point, users can see at a glance an indication of a characteristic of the actual flow being provided to determine e.g. how far from the desired set point the provided flow may be, and ascertain other useful information as will be explained below in the context of one application which includes the use of a cannula with a collapsible portion and bag mask ventilation.

The respiratory apparatus is controlled to provide a flow of gas according to the one or more displayed flow parameter settings, or set points, as shown on the display view. If the set points are not met, the display view changes to show the received values which may be the values measured by one or more sensors in the system. For example, if the selected flow rate value (set point) is 40 LPM and the flow supplied by the flow generator is below 40 LPM, the controller detects this and controls the display view to show the received value representing the flow rate being supplied by the flow generator (actual flow rate) instead of the selected flow rate value (flow rate set point). The value of the flow rate being supplied by the flow generator may be measured by sensors within the system, for example a flow sensor in fluid communication with the flow generator. The controller may control the display view to revert to presenting the selected flow rate value (set point) when the flow generator is supplying the flow at the selected value (actual flow rate meets set point). In another example, if the selected FiO2 is 100% and the flow supplied by the respiratory apparatus is below 100% FiO2, the controller detects this and controls the display view to show the received value representing the FiO2 being supplied by the respiratory apparatus (actual FiO2) instead of the selected FiO2 value (FiO2 set point). The value of the FiO2 being supplied by the respiratory apparatus may be measured by sensors in the system or may be derived or estimated from values taken from flow sensors in the air and O2 paths within the respiratory apparatus, and knowledge of the FiO2 set point. The controller may control the display view to revert to presenting the selected FiO2 (FiO2 set point) when the respiratory apparatus is supplying the flow at the selected value (actual FiO2 meets set point).

In some embodiments, the received values may be determined by the controller 19 from a signal received from one or more sensors sensing characteristics of the gases flow into or out of the respiratory apparatus. Signals may be received by controller 19 using wired or wireless techniques and may be electrical signals. The electrical signals may comprise a digital representation of the received values. Alternatively or additionally the received values may be derived by the controller from flow rate or pressure values ascertained from a flow generator or other component of the respiratory apparatus, or from the electrical current drawn by the flow generator which may be proportional to the flow rate generated. In some embodiments, the received values are flow parameters that are measured by equipment in the system 11. For example, the flow parameters may be measured by sensors such as sensors 18a, 18b, 18c, 18d, within the system 11. Alternatively or additionally one or more sensors may be placed in the in the gas path within the flow source 12. The flow source 12 may comprise a gas flow path for one or more of air, oxygen and mixed gases. One or more of these flow paths may comprise one or more flow and/or pressure sensors from which the received values may be determined by the controller 19. The controller 19 may use the received values, as may be determined or derived from the sensor signals, to determine the presence of a system generated trigger in response to which the controller causes the display view to switch.

In an example, the system-generated trigger may be a result of the change of state of the patient interface 200 from the first state to the second state which causes a change in flow parameters within the system 11. The change in flow parameters may cause one or more of the received values to cross a pre-determined threshold. As a result, the system-generated trigger is generated and in response the controller alters the display view to automatically replace presentation of the user-selected flow parameter value with the flow parameter value determined from the flow of gas. Flow parameters may be measured by sensors 18a, 18b, 18c, 18d, within the system 11. These sensors can provide the received values to the controller 19.

In some embodiments, the system-generated trigger may comprise one or more of the received values crossing a pre-determined threshold. For example, if the selected flow parameter set point is a flow rate such as 70 LPM selected e.g. by use of a quick select key comprising the third representation 430 (or using the slider 410), the trigger may comprise a threshold violation or crossing. The threshold may correspond to the selected flow rate setpoint value, or e.g. a deviation from the set point value, e.g. a percentage deviation as discussed below, or an absolute value deviation from the set point value such as e.g. +/−5 LPM, +/−10 LPM, +/−15 LPM or the like.

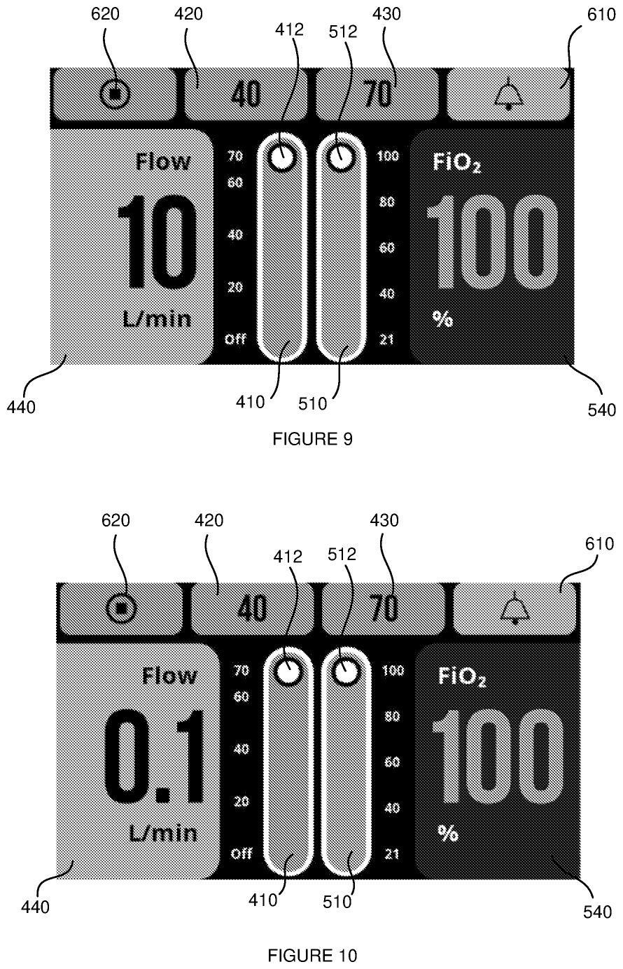

In a respiratory apparatus operating to provide a flow of gases in acceptable pressure and flow ranges, a threshold crossing may occur when the received values indicate the flow rate of the gases flow deviates by at least about +/−10% of the flow rate set point, i.e. crossing a threshold at about 77 LPM or about 63 LPM. In a respiratory apparatus operating to provide a flow of gases in a flow or pressure controlled state, a threshold crossing may occur when the received values indicate an operating flow rate that deviates by at least about +/−5% of the flow rate set point, i.e. crossing a threshold at about 73.5 LPM or about 66.5 LPM. When these threshold crossings occur, the conditions of a system-generated trigger are met and the user-selected flow parameter set point is automatically replaced in the display view with the flow rate value determined from the flow of gas. This is shown in FIG. 9 where the display region 440, which previously presented the set point value for flow rate, has been replaced with a value (e.g. 10 LPM) representing the flow rate in the gases flow. In some embodiments, replacement of the displayed values may be accompanied by an audible and/or visible alert (including activation of alarm element 610) as disclosed herein. In some embodiments, the displayed flow rate value determined from the flow of gas may be subsequently replaced with a warning view after a further threshold condition has been met. The further threshold condition may comprise a time threshold. A warning view may be presented when the displayed flow rate deviates from the flow rate set point for longer than a predetermined duration and/or when a second threshold value being a value corresponding to a more significant deviation from the setpoint, such as e.g. +/−20 lpm, +/−25 LPM or the like is met. A warning view may also be presented, indicating possible tube disconnection or leak in the gases flow path in the system.

In some embodiments, a feature of the display region 440, such as the brightness and/or contrast and/or colour and/or texture and/or animation (such as flashing or scrolling) may change when the system-generated trigger is received. For example, the background colour (as shown) or the alphanumeric text may change to a colour associated with alerts such as yellow (as shown), orange or red. Additionally, the alarm element 610 may become highlighted by flashing and/or colour change and/or presentation of an audible cue which in some examples, may be silenced by the user for a predetermined period. In some embodiments, when the received values indicate a flow rate less than 2 LPM, the controller may automatically alter the resolution of the displayed values to include the nearest first decimal point as shown in FIG. 10. In some embodiments, the display view preserves set point information intended for controlling operation of the flow source. For example, the selected flow rate remains visible by the location of control pointer 412 on slider 410 even during a condition when the system-generated trigger is received, and/or an alarm condition is met. Advantageously, ongoing presentation of the selected operational set point for the flow rate provides information to a user or maintenance person which may be useful to troubleshoot the device.

In some scenarios, displayed flow rate values may not represent the flow rate of gases reaching the patient. One such example is when the respiratory apparatus is being used with a non-sealing patient interface 15 which has a collapsible portion that is configured to be collapsed for example when a sealing face mask (e.g. bag mask) is applied over the top of the patient interface 15 to bag mask ventilate the patient. The non-sealing patient interface 15 comprises a non-sealing nasal cannula as shown in FIGS. 2, 24 and 25 and comprises nasal prongs that are configured to extend into the patient's nares in use. At least one of the prongs is sized to maintain a sufficient gap between the outer surface of the prong and the patient's skin to avoid sealing the gas path between the nasal cannula 15 and patient. Such an arrangement is disclosed in WO2022/130306 the entire contents of which are hereby incorporated herein by reference.

It may be useful to clinicians for flow rates to the patient interface to be displayed, especially during anaesthetic procedures for example, when they bag mask a patient. In an example, when the collapsible portion of the patient interface 15 collapses (e.g. when a mask is applied to the patient interface 15), the respiratory apparatus would provide 0 LPM to the patient interface 15. In another example, when the collapsible portion of the patient interface 15 collapses (e.g. when a mask is applied to the patient interface 15), the respiratory apparatus may provide some flows (e.g. at 5 or 10 LPM) to the patient interface 15. Using the presently disclosed switching of the display view content, the flow rate displayed when the patient interface 15 is in a collapsed state (i.e. the collapsible portion is collapsed), should provide feedback to the clinician of the level of collapse in the collapsible portion of the patient interface 15. For example, e.g. 0 LPM would indicate a full collapse while 5 LPM or 10 LPM would indicate a significant amount of collapse. This feedback presented by the display view when the system-generated trigger has been received by the controller provides validation of a blockage in the system which could result from the collapse of the collapsible portion of the patient interface 15. This can, for example, provide indication to a user that a bag mask has been appropriately used to collapse the collapsible portion of the patient interface 15 to reduce or stop a gas flow through the patient interface 15 to the patient. Advantageously, ongoing presentation of the selected operational set point for the flow rate provides information to a user to determine that collapse of the collapsible portion is in fact being achieved in the patient interface 15, and if the received flow rate value drops unexpectedly, to check if there is a system blockage or loose connection.

In some embodiments when there is a blockage in the system (i.e. in the respiratory apparatus and/or components downstream of the respiratory apparatus), for example when a collapsible portion of a nasal cannula is collapsed, the respiratory apparatus may be controlled to operate in a pressure-controlled mode. In this mode, the respiratory apparatus is controlled to limit pressure in the system. Control of the respiratory apparatus in this mode will modulate output pressure and/or flow based on predetermined pressure threshold values. In such a mode of operation, the controller may, upon detection of a flow rate threshold crossing, control the display view to switch from presenting the flow rate set point to presenting the received flow rate value. In this embodiment, the threshold may be +/−5% of the flow rate set point. Switching off the display view may be accompanied by an audible and/or visible alert as disclosed herein. In some modes, e.g. the pressure-controlled mode, flow rate related notifications and/or alerts and/or prompts (such as those described in the context of method 800) may take higher priority compared to notifications and/or alerts and/or prompts relating to other flow parameters. Flow rate related notifications and/or alerts and/or prompts can notify a user that respiratory support to a patient is becoming sparse or is being stopped. Notifications and/or alerts and/or prompts that relate to other flow parameters such as gas concentration may be of lesser significance in the clinical context. In some scenarios, notifications and/or alerts and/or prompts relating to non-flow rate parameters may be disabled or not presented when a flow rate alert has greater significance. For example a low FiO2 alert may not be presented when there is a low flow rate notification because in a zero or low flow situation gas concentration information may not be meaningful.

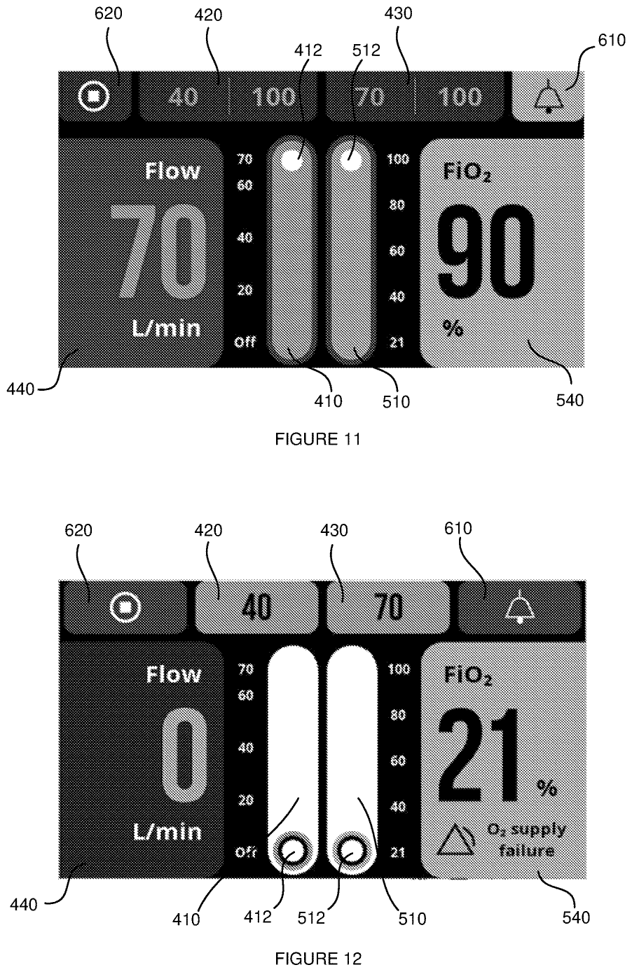

In another embodiment, the selected the flow parameter comprises Fraction of Inspired Oxygen (FiO2), selected by use of e.g. the slider 510, and the system-generated trigger may comprise a threshold crossing. A threshold crossing may occur when the received values indicate FiO2 deviates from the FiO2 set point by +/−5%. When a threshold crossing occurs, the conditions of a system-generated trigger are met and the user-selected FiO2 set point is automatically replaced with the received FiO2 value determined from the flow of gas. This is shown in FIG. 11, where the display region 540, which previously presented the set point for the FiO2, has been replaced with a value representing the FiO2 in the gases flow which in the example shown, is 90%. The system-generated trigger could be registered by the controller receiving a signal from one or more of e.g. a gas concentration sensor detecting gas concentration in the gases flow path anywhere within or downstream of the respiratory apparatus to the patient and/or changes in inlet O2 pressure determined by an O2 sensor or flow sensor in the oxygen flow path in the respiratory apparatus and/or by flow sensors. In some embodiments, the respiratory apparatus may derive or estimate a value of FiO2 in the gases flow from values taken from flow sensors in the air and O2 path within the respiratory apparatus, and knowledge of the FiO2 set point selected by the user. This derived or estimated FiO2 value may be used by the controller 19 to determine if there has been a threshold crossing corresponding to a system-generated trigger.

In some embodiments, a feature of the display region 540, such as the brightness and/or colour and/or contrast may change. For example, the background colour (as shown) or the alphanumeric text may change to a colour associated with alerts such as yellow (as shown), orange or red. Additionally, the alarm element 610 may become highlighted by flashing and/or colour change and/or presentation of an audible cue which in some examples, may be silenced by the user for a predetermined period. In some embodiments, the display view preserves set point information intended for controlling operation of the flow source. For example, the selected FiO2 remains visible by the location of control pointer 512 on slider 510 even during a condition when the system-generated trigger is received, and/or an alarm condition is met. Advantageously, ongoing presentation of the selected operational desired set point for the FiO2 provides information to a user or maintenance person which may be useful to troubleshoot the device, e.g. by checking the presence of and connection with the O2 supply. In some embodiments, the value displayed at display region 540 may be the actual FiO2 provided to the patient, measured by an Oxygen sensor at or downstream of the gases outlet of the respiratory apparatus.

In some embodiments, the system-generated trigger causing switching of the display view from “set point” values for a flow parameter to received values comprises a mis-match between received values representing the flow parameter and a pre-determined relationship or threshold stored in a memory device associated with the controller. For example, a memory device associated with the controller may store a set of data values or a function or look up table relating a range of set point flow rate values with expected pressure values. If, for a given flow rate set point, pressure exceeds the expected pressure value, the controller may switch the display view to show the received flow rate values. This may be useful since an unexpected increase in resistance to flow (which may be associated with an increase in pressure) may indicate that an incompatible patient interface has been connected. Alternatively or additionally, if for a given flow rate set point, received values for pressure are lower than expected, the controller may switch the display view to show the received flow rate values and/or provide a notification indicating a possible tube disconnection. This may be useful since a decrease in resistance to flow (which may be associated with a drop in pressure) may indicate a tube disconnection and/or leak in the gases flow path in the system. The switching of display views associated with the system-generated triggers may inform a user to check for unsafe use of the respiratory apparatus and/or patient interface and may in some embodiments be accompanied by a visible and/or audible alert presented by the I/O interface 20.

In addition to or as an alternative to the examples provided above, a system-generated trigger could be registered by the controller receiving a signal from one or more of e.g.:
a tachometer measuring a blower speed as a proxy for determining pressure;
a sensor measuring a proportional valve current used as a proxy for pressure;
changes in inlet pressure detected by an O2 pressure sensor at the respiratory apparatus inlet; and
a flow rate sensor measuring flow rates from an O2 flow path, air flow path (where a blower could reside) and/or mixed gas flow path of the respiratory apparatus.
a pressure sensor in the mixed gases flow path in the vicinity of the outlet where the conduit that delivers gases to the patient is connected.

In some embodiments, the system-generated trigger may comprise a physiological indicator determined by one or more instruments monitoring one or more patient condition parameters. The system-generated trigger could be registered by the controller receiving a signal from one or more of e.g.:
any sensor that measures blood gas parameters (SpO2, PaO2 etc.) which could provide an indirect indication that flow rate and/or FiO2 is not meeting the set point, and/or flow is not reaching the patient;
CO₂ sensors which could provide an indirect indication that a therapy is not being received by the patient. An increase in CO2 detected at the patient could indicate that one or more flow parameters are not meeting the set point and/or flow is not reaching the patient;
O₂ sensors which could provide an indirect indication that therapy is not being received by the patient. A decrease in O2 detected at the patient could indicate that one or more flow parameters are not meeting the set point and/or flow is not reaching the patient; and
a Gyro sensor that detects patient position and/or movement.

In some embodiments, the system-generated trigger may comprise a breathing interface indicator determined by instrumented detection of a mask placed over a nasal cannula providing a gases flow to a patient. The system-generated trigger could be registered by the controller receiving a signal from one or more instruments monitoring parameters such as e.g.:
acoustic parameters which could indicate breathing sounds within the interface, or measure reverb or reflection of sound waves;
proximity parameters which could indicate a mask and nasal interface are in close proximity to each other;
pressure parameters indicating pressure at e.g. a collapsible portion of the nasal cannula corresponding to application of a mask over the cannula;
gas concentration parameters indicating a change in gas concentration consistent with a change in the provision of gases to the patient by a nasal cannula or a mask; and
optical parameters sensing application of a mask over a collapsible nasal cannula.

In some embodiments, responsive to the controller receiving the system-generated trigger, the controller executes a delay routine before automatically causing the display view to replace the presented flow parameter setting with presentation of the received values. The display routine may delay automatic display view switching e.g. for 5, 10, 15, 20, 25 or 30 seconds. This may prevent unwanted display view switching resulting from spurious or transient conditions that inadvertently meet the conditions of the system-generated trigger.

In some embodiments, the display view 400 is operable to present warnings and alarm conditions to alert the user to an error condition in the respiratory apparatus. In one example shown in FIG. 12, absence of 02 supply entering the respiratory apparatus causes slider 510 to be disabled such that control pointer 512 cannot be used to select a FiO2 value higher than 21% corresponding to most room/ambient air. Additionally, the display view may alter the appearance, such as the colour, of one or elements of the view that relate to FiO2, such as the colour of slider 510 and/or control pointer 512, and the region 540 showing the parameter value. A colour associated with alerts may be preferred such as yellow, orange or red. Additionally and/or alternatively, further detail may be presented on the display view such as an image or text to indicate to the user that there is an O2 supply failure.

In some embodiments, if there is no O2 supply entering the respiratory apparatus, the device will default, on start-up, to operating at 21% FiO2 which is representative of the O2 concentration of most room air. An alert and/or a warning and/or user guidance to trouble shoot the O2 supply failure may be presented on the display view automatically upon system detection of disconnection of O2 gas supply and/or as a result of user interaction with the display view e.g. if the user attempts to change FiO2 to higher or lower than 21%. Meanwhile, alteration of the flow rate using the slider 410 of the first representation or the flow rate set point of the second representation 420 or the flow rate set point of the third representation 430 remains possible, albeit at FiO2 of 21% while the O2 supply failure exists. In some embodiments, alteration of the flow rate using the first, second and/or third representations 410, 420, 430 does not require a confirmation to be provided by the user, unless a flow rate of 0 LPM is selected in which case user confirmation will be prompted.

In some embodiments, when there is an O2 supply failure the display view may be modified to present a warning view such as the one shown in FIG. 13. The warning view may comprise e.g. a warning bar 630 across the top of the warning view containing e.g. the word “warning” and may flash and/or be presented in distinctive colour associated with alerts such as yellow, orange or red. An alarm element 610 may be provided which, in some examples, changes colour and/or flashes and/or is associated with an audible cue from the interface 20 to alert the user to the O2 supply failure. In some examples, alarm element 610 may be selectable by the user which upon selection, silences the audible cue for a predetermined period (e.g. 10 or 20 seconds). If the error condition is not resolved during the silenced period, the audible cue will be presented again. The warning view may guide the user to troubleshoot or check the oxygen supply to the respiratory apparatus with a visual and/or text guide. A prompt 670 may be provided to prompt the user to check if the oxygen supply is connected and supplying oxygen to the respiratory apparatus. When this occurs, the respiratory apparatus may continue to provide flows at a flow rate set point but with 21% FiO2 (corresponding to most room/ambient air). Once the oxygen supply is connected and supplying gases to the respiratory apparatus, the display view may return to present the selected flow parameters as they existed prior to the warning view of FIG. 13 appearing e.g. returning to a view as shown in the example of FIG. 6. This may occur automatically without the user interacting with the display view. Alternatively, the user can select the representation at region 660, acknowledging that there is an error and that the FiO2 is fixed at 21%. In this case, the respiratory apparatus may continue to provide flows at the flow rate set point previously set by the user. For example, if the device was providing 70 LPM prior to O2 supply loss, the warning view will revert to a display view which shows 70 LPM and 21% FiO2 as exemplified by FIG. 14. If desired the user may then select a new flow rate set point but with changes to 21% FiO2 disabled. This may cause presentation of a display view exemplified in FIG. 12, in which a user may change the flow rate set point. In this case the FiO2 slider may be disabled since there is no ability to increase the O2 content of the gas. The representation of the FiO2 value in display region 540 may be user-selectable such that upon selection, a view, such as the warning view in FIG. 13 appears, notifying the user that there is a fault with the O2 supply.

An O2 supply failure as discussed in relation to FIGS. 12 and 14 may be triggered when pressure sensors in an O2 flow path of the respiratory apparatus, for example at or upstream of the O2 inlet of the respiratory apparatus detect a pressure below a threshold or a threshold range such as about 220 kPa to about 250 kPA.

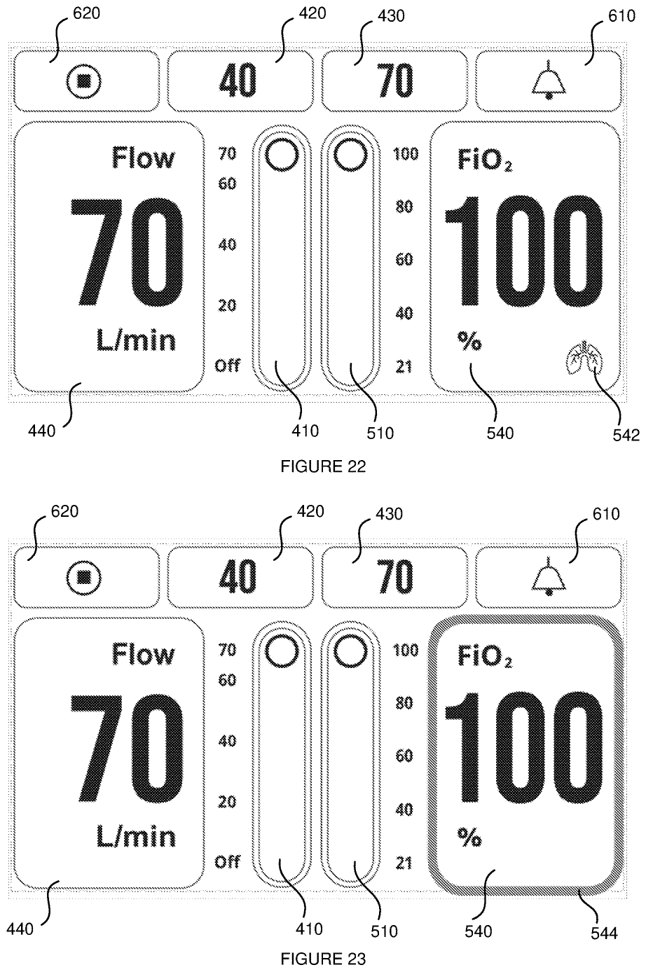

In some embodiments, the display view may be modified by the controller to provide a visible and/or audible indication that a FiO2 value higher than 21% has been selected for provision to the patient. The visible indication may comprise changing the appearance, such as the colour of the alphanumeric content and/or background or outline of the display region 540. Providing a visible and/or audible indication when a higher FiO2 value is selected may be useful to draw the user's attention to the presence of high concentration O2 which may have particular importance in scenarios involving use of equipment such as cauterisers and lasers and other devices that may present an ignition risk in the presence of high concentration 02. In some examples, it may be desirable that the changed appearance associated with display region 540 involves use of a colour other than red, orange or green which are associated with a stop, caution or proceed “traffic light” code. In some examples, purple, magenta, pink or other colours not used elsewhere in the display view may be suitable colours, to name a few. Alternatively or additionally, the visible indication may comprise one or more of changing the background colour of the display region 540 and optionally display region 440, presentation on the display view of an icon 542 in or near the display region 540 as shown in FIG. 22, and adding an outline 544, such as a coloured outline, to display region 540 as shown in FIG. 23. In some embodiments the visible indication may comprise an animation such as flashing or scrolling of the selected FiO2 value, optionally with a text such as e.g. “HIGH O2” or the like. The visible indication altering the appearance of the display region 540 may be triggered by selection of a FiO2 value higher than 21%, such as 25%, 30% or 35% or higher. The FiO2 value at which the visible alteration of the display region 540 is triggered may be configurable by the user.

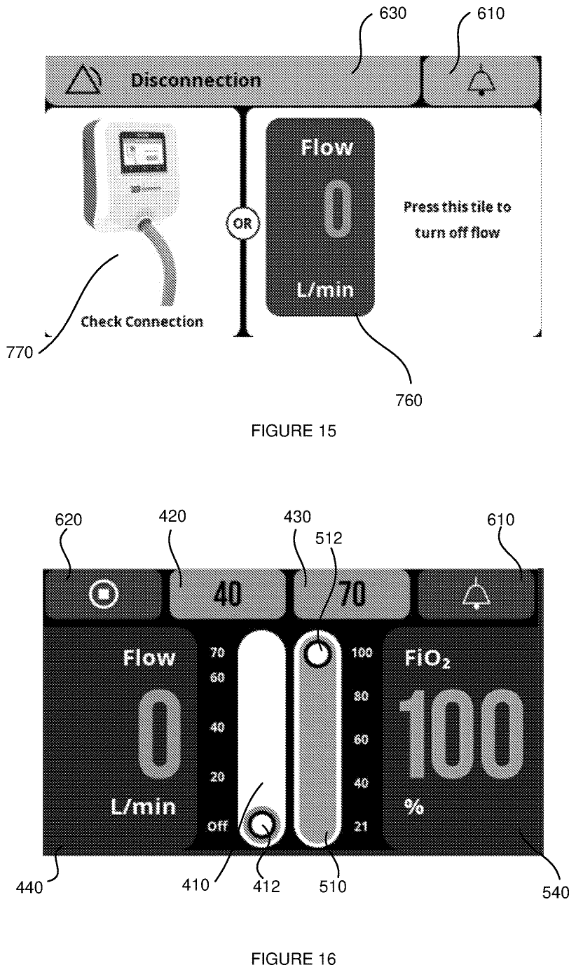

In an example shown in FIG. 15, a disconnection in the respiratory system (e.g. one or more pneumatically connected components of system 10 become disconnected) may cause presentation of a warning view. A warning view may comprise a warning bar 630 across the top of the warning view containing e.g. the word “disconnection” and may flash and/or be presented in a distinctive colour associated with alerts such as yellow, orange or red. An alarm element 610 may be provided which, in some examples, changes colour and/or flashes and/or is associated with an audible cue from the interface 20 to alert the user to the disconnection. In some examples, alarm element 610 may be selectable by the user which upon selection, silences the audible cue for a predetermined period (e.g. 10 or 20 seconds). If the error condition is not resolved during the silenced period, the audible cue will be presented again. The warning view may contain an image or text to indicate to the user that there is a disconnection in the respiratory system. The warning view may guide the user to check the connections in the system with a visual and/or text guide. A prompt 770 may be provided to prompt the user to check if the system components are properly connected. The display view may return to present the selected flow parameters upon rectification of the error. Alternatively or if the patient connection fault cannot be resolved, the user can select the representation at region 760, acknowledging that there is an error, controlling the apparatus to stop the flow (i.e. reducing flow to 0 LPM). FIG. 16 is an example of a display view that may follow confirmation via prompt 760, presenting the user-selected the operational parameters.

A disconnection error as discussed in relation to FIG. 15 may be triggered when e.g. at a given flow rate set point, system pressure measured by the respiratory apparatus significantly reduces and/or is low, or relationship of flow and pressure changes such that it exceeds a threshold.

Other errors arising due to faults that disrupt the respiratory apparatus from providing gases at user selected set points may be presented on the display screen in various warning views. Upon detection of these faults, it may be desirable for the controller to control operation of the respiratory apparatus in a “safe state” which may require the controller to automatically cut power to the flow generating elements (e.g. blower, proportional valve). Such warning views may comprise alarm identifiers and/or descriptors and/or troubleshooting guides.

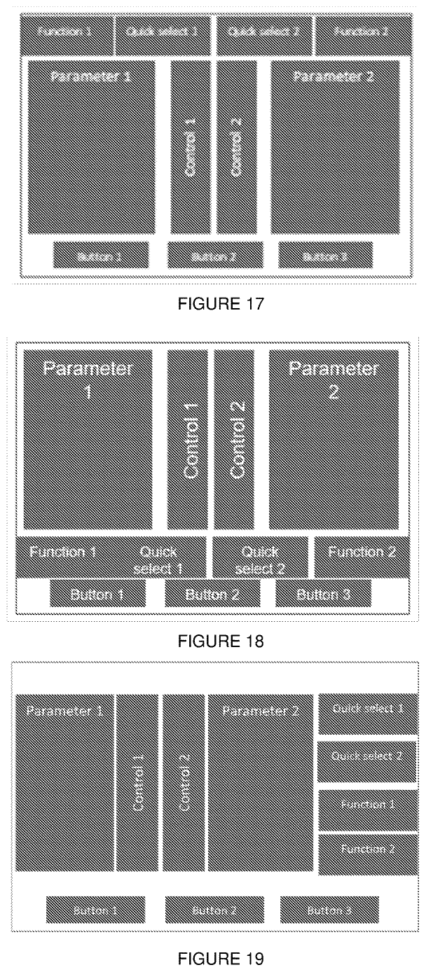

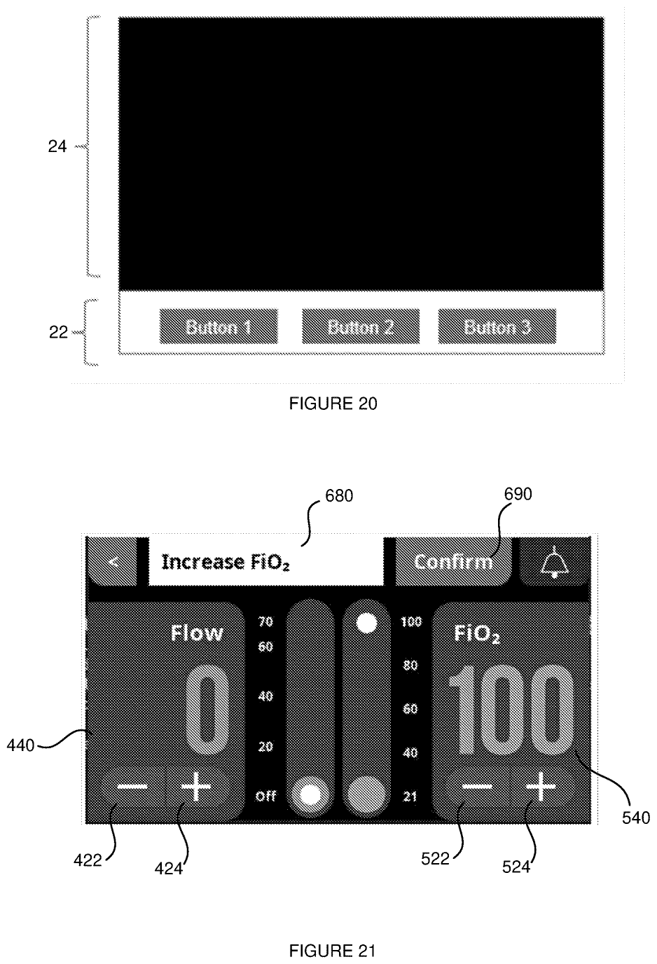

It is to be appreciated that the present disclosure is directed to functional and technical aspects pertaining to interactive user interfaces embodied in views presented on a display screen for the control of a respiratory apparatus. While examples have been provided in FIGS. 4 to 7, 9 to 16 and 23, it is to be understood that the embodiments disclosed herein are not limited to the specific layouts exemplified, and that other layouts are contemplated and expressly form part of this disclosure examples of such alternative layouts are exemplified in FIGS. 17 to 19, in which:
Control 1 and Control 2 are user-selectable representations (e.g. 410, 510) of flow parameters presenting a range of values,
Quick select 1 and Quick select 2 are user-selectable representations (e.g. 420, 430) of flow parameters, each presenting a single set point value,
Parameter 1 and Parameter 2 are display regions (e.g. 440, 540) for displaying either a selected parameter value (i.e. set point) or a received value for the parameter determined for the flow of gases if a system-generated trigger has been received.
Function 1 and Function 2 are user-selectable representations (e.g. 610, 620) of functional elements such as “stop” and “alert” elements as disclosed herein, Flow parameters may comprise e.g. flow rate or FiO2 consistent with the examples provided. In some embodiments, it may be preferred that the display regions for Parameter 1 and Parameter 2 enable the values of those parameters to be emphasised relative to other elements of the display view. For example, the amount of the display view area occupied by the display regions for Parameter 1 and Parameter 2 may be more than about 25% to about 50% of the display view height, for ease of visibility. In some embodiments, Button 1, Button 2 and Button 3 may be provided to give quick access to functions such as power on/off, log retrieval, and graphical display of therapy parameters. Button 1, Button 2 and Button 3 may comprise user-selectable elements on the display view in a manner similar to the Control, Quick select, Parameter and Function keys. Thus, they may be transient buttons not visible when the display screen is powered down. Alternatively, one or more of these buttons may be non-transient physical buttons mounted on the display device and visible/selectable by touch even when the display screen is powered down. These buttons may have labels physically printed and/or etched/embossed into them so that they are always visible, even while the display screen is off. One or more of these buttons may be arranged along a bottom, top or side edge of the display device. The schematic drawing of FIG. 20 shows one example, with Button 1, Button 2 and Button 3 each provided as a physical button on the display device 22, beneath a touch sensitive display screen 24 shown in an off configuration.

Notwithstanding improvements enabled by the present disclosure in relation to quick select keys, it is to be noted that controls to adjust the flow parameters, including flow rate and FiO2 remain on the display view throughout. The controls may be in the form of sliders that move in increments of e.g. 10 LPM or 20% FiO2. In some embodiments, the sliders may be positioned adjacent to each other on the display view so that both parameters can be altered simultaneously using 2 fingers (i.e. multi-touch control). Finer control is also possible as shown in the display view of FIG. 21 which includes pairs of +/− symbols 422, 424 and 522, 524. In some embodiments, finer control symbols 422, 424 and 522, 524 are normally not visible on the display view and may appear only when the user selects the display region e.g. 440, 540 of the parameter they wish to adjust. Finer adjustments of e.g. 1 unit may be made using the +/− symbols than the sliders 410, 510.

Symbols 422, 424 and 522, 524 may disappear from the display view after a predetermined time of inactivity during which no user selections are made, or after the flow parameter has been selected and a confirmation input provided if prompted. In some examples, symbols 422, 424 do not appear on the display view when the display region 440 is not active. In some examples, region 440 is user selectable and upon selection of region 440, the area becomes “active” and symbols 422, 424 appear. Alternatively or additionally symbols 422, 424 may appear when the user has positioned the slider pointer 412 to select a flow rate of 0 LPM. In some examples, symbols 422, 424 may fade and/or become disabled or vanish or not appear on the display view while the slider 410 is activated by operation of the slider pointer 412 to select a non-zero value. In some embodiments, symbols 522, 524 do not appear on the display view when the display region 540 is not active (e.g. for symbols 522, 524 when there is an O2 connection error and FiO2 cannot be altered from 21%). In some examples, region 540 is user selectable and upon selection of region 540, the area becomes “active” and symbols 522, 524 appear. In some examples, symbols 522, 524 appear on the display view when the slider 510 is activated by operation of the slider pointer 512 to select a non-zero value. In some examples, symbols 522, 524 may fade and/or become disabled or vanish or not appear on the display view while the slider 510 is activated by operation of the slider pointer 512 to select a non-zero value.

In some embodiments, adjustment of the parameter value/set point may cause the controller to present a notification of change and request confirmation before altering control of the respiratory apparatus to the adjusted value. In the example shown in FIG. 21, FiO2 has been increased to 100% and notification 680 has been presented showing the message “Increase FiO2” with user-selectable confirmation element 690. Although not shown, if the user adjusts the parameter set point from a higher value to a lower value, notification 680 can appear with a message indicating a decrease in the parameter, along with confirmation element 690. Upon selection of confirmation element 690, therapy commences at the confirmed set point. It is to be noted however that the notification and confirmation elements may not be essential, particularly in respect of adjustments to flow rate. In some embodiments, one or more selectable elements corresponding to quick select keys may be provided for quick selection of a desired FiO2 for the flow of gases. For example, quick select key may be provided for 21% FiO2 and for 100% FiO2 although these values are examples only and other values may be provided such as 30%, 35%, 40%, . . . 85%, 90%, 95%, 100%. In some embodiments, the value associated with FiO2 quick select keys may be configurable by a user who may prefer particular FiO2 parameters for “quick selection”. Configuration may be achieved by the user making user preference selections using the I/O interface 20. In some embodiments, upon start-up the controller 19 may cause the display device to prompt the user to supply an identification input that enables the controller to retrieve pre-configured user preference selections for that user. In cases where manufacturer and/or regulatory standards require a confirmation input to change the FiO2 set point, the quick select keys may require a 2-step process wherein the selection of the value corresponding to the quick select key may subsequently require confirmation input from the user (e.g. as described in relation to FIG. 21). Provision of FiO2 quick select keys may be in addition to or an alternative to slider 510 and/or symbols 522,524.

It is to be appreciated that the present disclosure may be embodied as a method, system, computer program product or a computer-readable memory device. The computer-readable memory device may comprise a computer-readable signal medium or a computer-readable storage medium. A computer-readable medium may comprise, for example, but is not limited to, any physical device or material that can store digital data, such as a hard disk, CD-ROM, flash drive, an electronic, magnetic, optical, electromagnetic, infrared, solid state or semiconductor system, apparatus, or device, or any suitable combination of these. Thus, the present disclosure may provide a non-transitory data carrier carrying code which, when implemented on a controller, causes the controller to carry out any of the methods described herein.

It is to be understood that the examples discussed in relation to the display screens and display views of FIGS. 4 to 7 and 9 to 23 are presented herein in the context of explaining aspects of the disclosure comprising a method of controlling a respiratory apparatus, as well as a system for controlling a respiratory apparatus such as, but not limited to, the system discussed in relation to FIG. 1. Similarly, it is to be understood that these examples are illustrative of the effect of a computer program product embodied on a memory device containing instructions causing a controller having an associated display screen to perform methods of this disclosure. It is to be understood that the memory device may form part of the controller 19 as illustrated in FIG. 1. Alternatively, the memory device may comprise a storage device that is accessible by the controller 19. Such a memory device may be local to the respiratory apparatus, such as part of the respiratory apparatus, or it may be remotely located and accessible via one or more wired or wireless networks.

Quick select keys may be useful in a fast paced environment such as in an operating theatre. The use of quick select keys as disclosed herein may enable the user to accurately provide the flow required to the patient without having to move a slider or dial (which requires precision) or make selections through multiple display views and menu selections to change and confirm flow rates. This leaves the clinician with more time to spend caring for the patient rather than interacting with the interface controlling the respiratory apparatus. This is in contrast to existing devices e.g. ventilators, anaesthesia machines or other flow generating devices where a user is asked to confirm a parameter change. This makes the process of changing flowrate a tedious one. Moreover, in some existing devices, selection of different flow parameters can require multiple interactions with the control interface such as via menus and different screen views, confirmation prompts and the like in order to change a pre-set parameter.

Additionally, presenting on a display view values representing parameters such as the flow rate and/or FiO2 of gases in the flow path in circumstances when a system-generated trigger has been received provides richer and more timely clinical data. This empowers clinicians to rapidly evaluate clinically important factors such as e.g. the condition of the gases flow into and/or out of the respiratory apparatus, the arrangement of a mask over a collapsible portion of a patient interface while on the patient and the condition of the patient to name a few.

Where the terms “comprise”, “comprises”, “comprised” or “comprising” are used in this specification (including the claims) they are to be interpreted as specifying the presence of the stated features, integers, steps or components, but not precluding the presence of one or more other features, integers, steps or components or group thereof.

It is to be understood that various modifications, additions and/or alterations may be made to the parts previously described without departing from the ambit of the present invention as defined in the claims appended hereto.

Future patent applications may be filed on the basis of or claiming priority from the present application. It is to be understood that the following claims are provided by way of example only, and are not intended to limit the scope of what may be claimed in any such future application. Features may be added to or omitted from the claims at a later date so as to further define or re-define the invention or inventions.

Alternative aspects are set out in the following clauses:
1. A method for operating a respiratory apparatus, comprising the steps of:
   presenting on a display screen a display view comprising:
      a user-selectable first representation of a flow parameter, the first representation enabling selection from a range of flow parameter values; and
      a user-selectable second representation of the flow parameter, the second representation comprising a single flow parameter set point value;
   receiving a user selection of the first representation or the second representation; and
   responsive to the user selection, controlling the respiratory apparatus to provide a gases flow according to the parameter value corresponding to the selected first representation or the selected second representation.
2. The method according to clause 1, wherein the user-selectable first representation and the user-selectable second representation are discrete representations.
3. The method according to any one of the preceding clauses, wherein the user-selectable first representation and the user-selectable second representation are presented on the display view substantially simultaneously.
4. The method according to any one of the preceding clauses, wherein the respiratory apparatus comprises at least one of a blower and a valve, and wherein the method comprises controlling the blower and/or the valve to provide the gases flow according to the parameter value corresponding to the selected first representation or the selected second representation.
5. The method according any one of the preceding clauses, wherein the user selection of the first representation or the second representation is received via the display screen, and the respiratory apparatus is controlled to provide the gases flow according to the parameter value corresponding to the selected first representation or the selected second representation.
6. The method according to any one of the preceding clauses, wherein the gases flow is provided to a patient via a non-sealing interface that is in fluid communication with an outlet of the respiratory apparatus.
7. The method according to any one of the preceding clauses, wherein the flow parameter comprises flow rate.

8. The method according to clause 7, comprising providing the gases flow at a flow rate of at least about 15 LPM.
9. The method according to any one of the preceding clauses, wherein the second representation comprises a flow rate set point value selected from the group comprising 40 LPM and 70 LPM.
10. The method according to any one of the preceding clauses, comprising presenting on the display view a third user-selectable representation of the flow parameter, the third representation comprising a single flow parameter set point value different from the set point value of the second representation.
11. The method according to clause 10, wherein the second representation and the third representation each comprise a single input selector.
12. The method according to clause 10 or clause 11, wherein the third representation comprises a flow rate set point value selected from the group comprising 40 LPM and 70 LPM.
13. The method according to any one of clauses 10 to 12, wherein the third representation is a discrete representation from the first and second representations.
14. The method according to any one of clauses 10 to 13, wherein the third representation is presented substantially simultaneously with the first and second representations.
15. The method according to any one of the preceding clauses, wherein the first representation comprises one or more of a slider; a dial; up/down arrows; +/− symbols; other symbols representing an increase or decrease to the flow parameter value within the range.
16. The method according to any one of the preceding clauses wherein the display screen comprises a touch screen.
17. The method according to any one of the preceding clauses wherein the user selections of the representations presented in the display view are received by a controller which, responsive to the user selections, controls the respiratory apparatus.
18. The method according to any one of the preceding clauses, comprising upon startup of the apparatus, a controller setting a flow parameter for the gases flow to a predetermined oxygen concentration value.
19. The method according to clause 18, comprising, responsive to the controller receiving a user selection of a flow rate value, controlling the respiratory apparatus to provide the gases flow at the predetermined oxygen concentration value and the selected flow rate value.
20. The method according to clause 18 or clause 19, wherein the predetermined oxygen concentration value is 21% FiO2 or 100% FiO2.
21. The method according to any one of the preceding clauses, wherein the controller does not prompt the user for a confirmation input when controlling the respiratory apparatus to provide a gases flow according to a flow rate value corresponding to user selection of any one of the first representation, the second representation and the third representation.

1. A method for operating a respiratory apparatus, comprising the steps of:
presenting on a display screen a display view comprising:
a user-selectable first representation of a flow parameter, the first representation enabling selection from a range of flow parameter values; and
a user-selectable second representation of the flow parameter, the second representation comprising a single flow parameter set point value;
receiving a user selection of the second representation; and
responsive to the user selection, controlling the respiratory apparatus to provide a gases flow according to the parameter value corresponding to the second representation.

2. The method according to claim 1, wherein the user-selectable first representation and the user-selectable second representation are discrete representations.

3. The method according to any one of the preceding claims, wherein the user-selectable first representation and the user-selectable second representation are presented on the display view substantially simultaneously.

4. The method according to any one of the preceding claims, wherein the respiratory apparatus comprises at least one of a blower and a valve, and wherein the method comprises controlling the blower and/or the valve to provide the gases flow according to the parameter value corresponding to the second representation.

5. The method according any one of the preceding claims, wherein the user selection of the second representation is received via the display screen, and the respiratory apparatus is controlled to provide the gases flow according to the parameter value corresponding to the second representation.

6. The method according to any one of the preceding claims, wherein the gases flow is provided to a patient via a non-sealing interface that is in fluid communication with an outlet of the respiratory apparatus.

7. The method according to any one of the preceding claims, wherein the flow parameter comprises flow rate.

8. The method according to claim 7, comprising providing the gases flow at a flow rate of at least about 15 LPM.

9. The method according to any one of the preceding claims, wherein the second representation comprises a flow rate set point value selected from the group comprising 40 LPM and 70 LPM.

10. The method according to any one of the preceding claims, comprising presenting on the display view a third user-selectable representation of the flow parameter, the third representation comprising a single flow parameter set point value different from the set point value of the second representation.

11. The method according to claim 10, wherein the second representation and the third representation each comprise a single input selector.

12. The method according to claim 10 or claim 11, wherein the third representation comprises a flow rate set point value selected from the group comprising 40 LPM and 70 LPM.

13. The method according to any one of claims 10 to 12, wherein the third representation is a discrete representation from the first and second representations.

14. The method according to any one of claims 10 to 13, wherein the third representation is presented substantially simultaneously with the first and second representations.

15. The method according to any one of the preceding claims, wherein the first representation comprises one or more of a slider; a dial; up/down arrows; +/− symbols;
other symbols representing an increase or decrease to the flow parameter value within the range.

16. The method according to any one of the preceding claims wherein the display screen comprises a touch screen.

17. The method according to any one of the preceding claims wherein the user selections of the representations presented in the display view are received by a controller which, responsive to the user selections, controls the respiratory apparatus.

18. The method according to any one of the preceding claims, comprising upon startup of the apparatus, the controller setting a flow parameter for the gases flow to a predetermined oxygen concentration value.

19. The method according to claim 18, comprising, responsive to the controller receiving a user selection of a flow rate value, controlling the respiratory apparatus to provide the gases flow at the predetermined oxygen concentration value and the selected flow rate value.

20. The method according to claim 18 or claim 19, wherein the predetermined oxygen concentration value is 21% FiO2 or 100% FiO2.

21. The method according to any one of the preceding claims, wherein the controller does not prompt the user for a confirmation input when controlling the respiratory apparatus to provide a gases flow according to a flow rate value corresponding to user selection of any one of the first representation, the second representation and the third representation.

22. A system for operating a respiratory apparatus according to the method of any one of the preceding claims.

23. A system for operating a flow source, the system comprising:
a display screen operable to present a display view comprising:
a user-selectable first representation of a flow parameter, the first representation enabling selection from a range of flow parameter values; and
a user-selectable second representation of the flow parameter, the second representation comprising a single flow parameter set point value; and
a controller for receiving user selections, and responsive to a user selection of the first representation or the second representation, controlling a flow source to provide the gases flow according to the parameter value corresponding to the selected first representation or the selected second representation.

24. The system of claim 23, comprising the flow source.

25. The system of claim 24, wherein the flow source comprises at least one of a blower and a valve and the controller controls the blower and/or the valve to provide the gases flow according to the parameter value corresponding to the first representation or the second representation.

26. The system according to any one of claims 23 to 25, wherein the user selection of the first representation or the second representation is received at the controller via the display screen, and the controller controls the flow source to provide the gases flow according to the parameter set point value corresponding to the first representation or the second representation.

27. The system according to any one of claims 23 to 26, wherein the gases flow is provided to the patient via a non-sealing interface that is provided in fluid communication with an outlet of the flow source.

28. The system according to any one of claims 23 to 27, wherein the flow parameter comprises flow rate.

29. The system according to any one of claims 23 to 28, wherein the controller controls the flow source to provide the gases flow at a flow rate of at least about 15 LPM.

30. The system according to any one of claims 23 to 29, wherein the second representation comprises a flow rate set point value selected from the group comprising 40 LPM and 70 LPM.

31. The system according to any one of claims 23 to 30, comprising presenting on the display view a third user-selectable representation of the flow parameter, the third representation comprising a single flow parameter set point value different from the set point value of the second representation.

32. The system according to claim 31, wherein the second representation and the third representation each comprise a single input selector.

33. The system according to claim 31 or claim 32, wherein the third representation comprises a flow rate set point value selected from the group comprising 40 LPM and 70 LPM.

34. The method according to any one of claims 31 to 33, wherein the third representation is presented substantially simultaneously with the first and second representations.

35. The system according to any one of claims 23 to 34, wherein the first representation comprises one or more of a slider; a dial; up/down arrows; +/− symbols; other symbols representing an increase or decrease to the flow parameter value within the range.

36. The system according to any one of claims 23 to 35, wherein the display screen comprises a touch screen.

37. The system according to any one of claims 24 to 36, wherein the flow source comprises one or more gases inlets.

38. The system according to any one of claims 23 to 37, wherein the system comprises one or more gas outlets for fluid communication, with a conduit configured to provide the gases flow to the patient.

39. The system according to claim 38, wherein the one or more gas outlets are couplable with:
a conduit configured to provide the gases flow to a patient interface.

40. The system according to any one of claims 23 to 39, wherein the system comprises a humidifier.

41. The system according to any one of claims 23 to 40, wherein the system is operable to control a flow source provided in one or both of:
a separate device; and
a respiratory apparatus incorporating the system comprising the display screen and the controller.

42. The system according to any one of claims 23 to 41, wherein the system comprises part of an anaesthesia machine with capability to provide high flow gases, and wherein the controller controls the gases flow from the anaesthesia machine according to user selections of one or more flow parameter values received by the controller.

43. The system according to any one of claims 23 to 42, wherein the system comprises part of a respiratory apparatus providing the gases flow according to user selections of one or more flow parameter values received by the controller.

44. The system according to any one of claims 23 to 41, wherein the system comprises part of an anaesthesia machine or ventilator, and wherein the controller is operatively couplable with a respiratory apparatus separate from the anaesthesia machine or ventilator and which is operable to provide the gases flow according to user selections of one or more flow parameter values received by the controller.

45. The system according to claim 44, wherein the anaesthesia machine or ventilator is operatively couplable with the respiratory apparatus by one or more of a wireless, wired or contact coupling.

46. The system according to claim 45, wherein the anaesthesia machine or ventilator comprises a physical interface configured to cooperate with the respiratory apparatus.

47. The system according to claim 46, wherein the physical interface comprises a recess for receiving at least part of the respiratory apparatus.

48. The system according to any one of claims 23 to 47, wherein upon startup of the system, the controller sets a flow parameter for the gases flow to a predetermined oxygen concentration value.

49. The system according to claim 48, wherein the controller, responsive to receiving a user selection of a flow rate value, controls the flow source to provide the gases flow at the predetermined oxygen concentration value and the selected flow rate value.

50. The system according to claim 48 or claim 49, wherein the predetermined oxygen concentration value is 21% FiO2 or 100% FiO2.

51. The system according to any one of claims 23 to 50, wherein the controller does not prompt the user for a confirmation input when controlling the respiratory apparatus to provide a gases flow according to a flow rate value corresponding to user selection of any one of the first representation, the second representation and the third representation.

52. A computer program product embodied on a memory device containing instructions for implementing, when executed by a controller, the method according to any one of claims 1 to 22.

53. A system for controlling a respiratory apparatus, the system comprising:
a display screen operable to present a display view comprising:
a representation of a flow parameter setting;
a controller for:
controlling the respiratory apparatus to provide the gases flow according to the flow parameter setting;
determining received values representing a flow parameter of the gases flow; and
responsive to receiving a system-generated trigger, automatically causing the display view to replace the presented representation of the flow parameter setting with presentation of the received values representing the flow parameter of the gases flow.

54. The system according to claim 53, wherein the system-generated trigger comprises the received values crossing a pre-determined threshold.

55. The system according to claim 53 or claim 54, wherein the pre-determined threshold is at least about +/−5% of the flow parameter setting.

56. The system according to any one of claims 53 to 55, wherein the pre-determined threshold is at least about +/−10% of the flow parameter setting.

57. The system according to claim 53, wherein the system-generated trigger comprises a mis-match between received values representing the flow parameter and a pre-determined relationship.

58. The system according to claim 57, wherein the system-generated trigger comprises a physiological indicator determined by one or more instruments monitoring one or more patient condition parameters.

59. The system according to claim 58, wherein the one or more patient condition parameters are selected from a group comprising:
a blood gas parameter;
an expired gas parameter;
an inspired gas parameter; and
patient position.

60. The system according to claim 53, wherein the system-generated trigger comprises a breathing interface indicator determined by instrumented detection of a mask placed over a nasal cannula providing a gases flow to a patient.

61. The system according to claim 60, wherein the breathing interface indicator is triggered by one or more instruments monitoring parameters selected from a group comprising:
acoustic parameters;
proximity parameters;
pressure parameters;
gas concentration parameters; and
optical parameters.

62. The system according to any one of claims 53 to 61, wherein the controller, responsive to receiving the system-generated trigger, automatically causes an audible device to present an audible and/or visible alert.

63. The system according to any one of claims 53 to 62, wherein the controller, responsive to receiving the system-generated trigger, automatically causes a colour and/or brightness change on at least a part of the display view.

64. The system according to any one of claims 53 to 63, wherein the received values represent the same flow parameter as the flow parameter setting.

65. The system according to any one of claims 53 to 64, wherein the flow parameter comprises flow rate.

66. The system according to any one of claims 53 to 65, wherein the flow parameter comprises Fraction of Inspired Oxygen (FiO2).

67. The system according to any one of claims 53 to 66, wherein the flow parameter comprises pressure of gases in the gases flow.

68. The system according to any one of claims 53 to 67, wherein responsive to the controller receiving the system-generated trigger, the controller executes a delay routine before automatically causing the display view to replace the presented flow parameter setting with presentation of the received values.

69. The system according to any one of claims 53 to 68, wherein the controller determines the system-generated trigger according to a mode of operation of the respiratory apparatus.

70. The method according to any one of claims 53 to 69, wherein the controller determines the system-generated trigger according to whether the respiratory apparatus is operating in a pressure-controlled mode or a flow-controlled mode.

71. The system according to any one of claims 53 to 70, wherein the controller determines the received values from signals received from one or more sensors and/or components of the respiratory apparatus.

72. The system according to any one of claims 53 to 71, comprising the respiratory apparatus, the respiratory apparatus comprising a flow source.

73. The system according to any one of claims 53 to 72, wherein the display screen comprises a touch screen.

74. The system according to any one of claims 53 to 73, wherein the system comprises one or more gases inlets to the respiratory apparatus.

75. The system according to any one of claims 53 to 74, wherein the system comprises one or more gas outlets for fluid communication with a conduit configured to provide the gases flow to the patient.

76. The system according to claim 75, wherein the one or more gas outlets are couplable with:
a conduit configured to provide the gases flow to a patient interface.

77. The system according to any one of claims 53 to 76, wherein the system comprises a humidifier.

78. The system according to any one of claims 53 to 77, wherein the system, is operable to control a flow source provided in one or both of:
a separate device; and
a respiratory apparatus incorporating the system comprising the display screen and the controller.

79. The system according to any one of claims 53 to 78, wherein the system comprises part of an anaesthesia machine with capability to provide high flow gases, and wherein the controller controls the gases flow from the anaesthesia machine according to the flow parameter setting.

80. The system according to any one of claims 53 to 78, wherein the system comprises part of a respiratory apparatus providing the gases flow according to the flow parameter setting.

81. The system according to any one of claims 53 to 78, wherein the system comprises part of an anaesthesia machine or ventilator, and wherein the controller is operatively couplable with a respiratory apparatus separate from the anaesthesia machine or ventilator and which is operable to provide the gases flow according to the flow parameter setting.

82. The system according to claim 81, wherein the anaesthesia machine or ventilator is operatively couplable with the respiratory apparatus by one or more of a wireless, wired or contact coupling.

83. The system according to claim 82, wherein the anaesthesia machine or ventilator comprises a physical interface configured to cooperate with the respiratory apparatus.

84. The system according to claim 83, wherein the physical interface comprises a recess for receiving at least part of the respiratory apparatus.
